(19) United States
(12) Patent Application Publication
(10) Pub. No.: US 2017/0145412 A1
(43) Pub. Date: May 25, 2017

(54) COMPOSITIONS FOR INHIBITING NLRP3 GENE EXPRESSION AND USES THEREOF

(71) Applicant: Idera Pharmaceuticals, Inc., Cambridge, MA (US)

(72) Inventors: Weiwen Jiang, Winchester, MA (US); Mallikarjuna R. Putta, Lexington, MA (US); Fu-Gang Zhu, Bedford, MA (US); Jillian M. DiMuzio, Harleysville, PA (US); Lakshmi Bhagat, Framingham, MA (US); Sudhir Agrawal, Shrewsbury, MA (US)

(21) Appl. No.: 15/342,054

(22) Filed: Nov. 2, 2016

Related U.S. Application Data
(60) Provisional application No. 62/250,796, filed on Nov. 4, 2015.

(51) Int. Cl. C12N 15/113; A61K 45/06; A61K 31/7088

(52) U.S. Cl. CPC C12N 15/113; A61K 31/7088; A61K 45/06; C12N 2310/11

(57) ABSTRACT

The present invention is directed to compounds, compositions, and methods useful for modulating NLRP3 mRNA or protein expression using gene silencing compounds comprising two or more single stranded antisense oligonucleotides that are linked through their 5′-ends to allow the presence of two or more accessible 3′-ends.

### RELATED APPLICATIONS

This application claims the benefit of U.S. provisional patent application Ser. No. 62/250,796, filed on Nov. 4, 2015, the contents of which are incorporated herein by reference in its entirety.

### BACKGROUND OF THE INVENTION

Field of the Invention

The present invention relates to compounds, compositions, and methods of use for the inhibition of NLR family, pyrin domain containing 3 (NLRP3; also known as CIAS1) gene expression or for diagnosing, treating and/or preventing diseases and/or conditions that respond to the inhibition of NLRP3 gene expression.

Summary of the Related Art

The NLRP3 gene belongs to a family of genes called NLR (nucleotide-binding domain and leucine rich repeat containing family). NLR proteins are involved in the immune system, helping to start and regulate the immune system's response to injury, toxins, or invasion by microorganisms. These proteins recognize specific molecules, become activated, and respond by helping to engage components of the immune system.

The NLRP3 gene provides instructions for making a protein called cryopyrin, which is found mainly in white blood cells and in cartilage-forming cells (chondrocytes). Cryopyrin recognizes bacterial particles; chemicals such as asbestos, silica, and uric acid crystals; and compounds released by injured cells.

Once activated, groups of cryopyrin molecules assemble themselves along with other proteins into structures called inflammasomes, which are involved in the process of inflammation. Inflammation occurs when the immune system sends signaling molecules as well as white blood cells to a site of injury or disease to fight microbial invaders and facilitate tissue repair.

Mutations in the NLRP3 gene are associated with familial cold autoinflammatory syndrome (FCAS), Muckle-Wells syndrome (MWS), chronic infantile neurological cutaneous and articular (CINCA) syndrome, and neonatal-onset multisystem inflammatory disease (NOMID). NLRP3 has also been implicated in the pathogenesis of interstitial cystitis/bladder pain syndrome (IC/BPS). Thus, there exists a need for treatments for diseases or disorders that would benefit from the reduced expression of NLRP3, or from modulation of NLRP3 activity.

### BRIEF SUMMARY OF THE INVENTION

The present invention is directed to compounds, compositions, and methods useful for modulating NLRP3 mRNA or protein expression using gene silencing compounds (“GSOs”) comprising two or more single stranded antisense oligonucleotides that are linked through their 5′-ends to allow the presence of two or more accessible 3′-ends. The gene silencing compounds according to the invention effectively inhibit or decrease NLRP3 mRNA or protein expression.

Provided herein are methods, compounds, and compositions for modulating expression of NLRP3 mRNA and protein. In certain embodiments, compounds useful for modulating expression of NLRP3 mRNA and protein are gene silencing compounds.

In certain embodiments, modulation can occur in a cell or tissue. In certain embodiments, the cell or tissue is in an animal. In certain embodiments, the animal is a human. In certain embodiments, NLRP3 mRNA levels are reduced. In certain embodiments, NLRP3 protein levels are reduced. Such reduction can occur in a time-dependent manner or in a dose-dependent manner.

Also provided are methods, compounds, and compositions useful for preventing, treating, and ameliorating diseases, disorders, and conditions.

In certain embodiments, methods of treatment include administering a NLRP3 mRNA or protein expression gene silencing compound or composition to an individual in need thereof.

### BRIEF DESCRIPTION OF THE FIGURES

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 depicts screening results of exemplary mNLRP3 GSOs in a cell based assay.

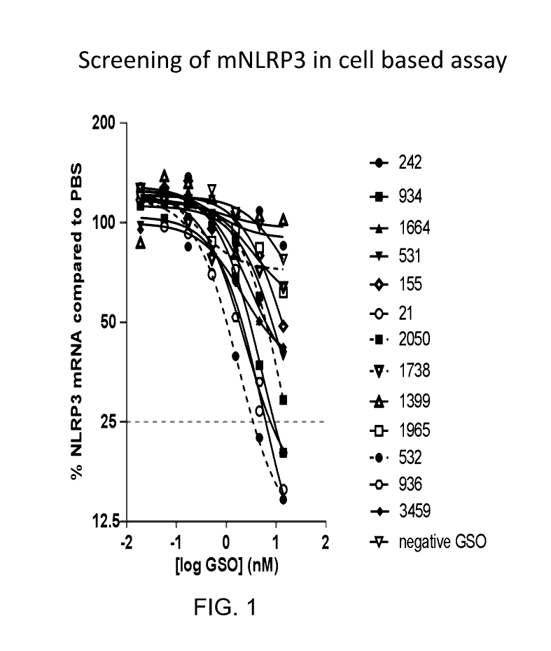

FIG. 2 depicts the silencing of mNLRP3 by exemplary GSOs in mouse J774 cell line.

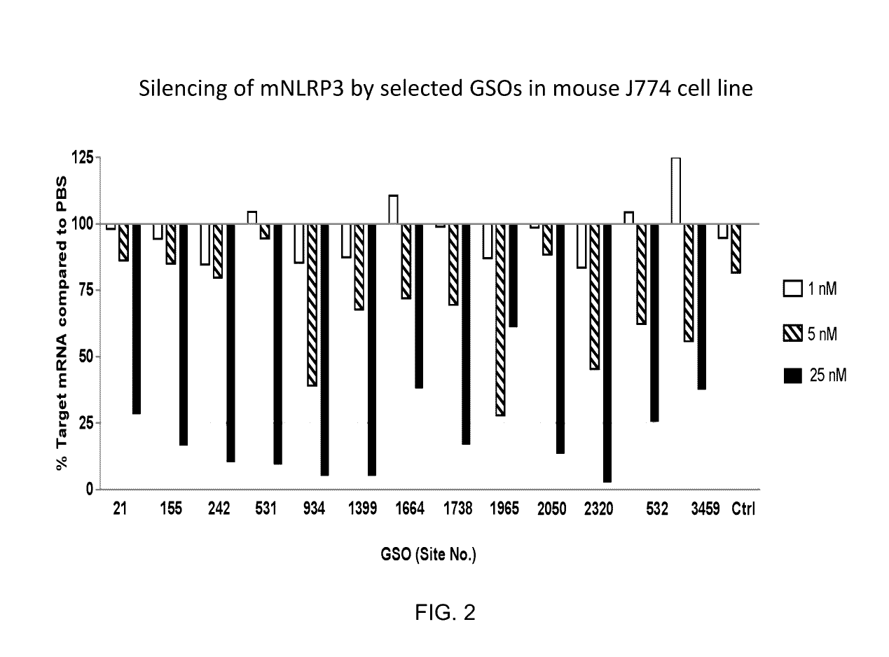

FIG. 3A and FIG. 3B demonstrate that exemplary mNLRP3 GSO dose dependently silences NLRP3 mRNA expression and protein levels in mouse J774 cells.

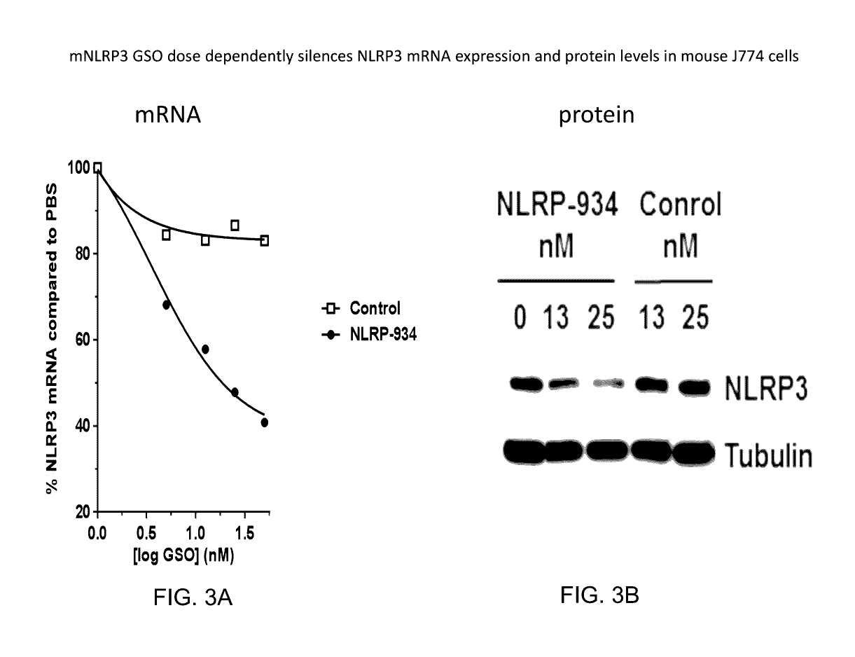

FIG. 4 demonstrates that exemplary mNLRP3 GSO dose dependently reduces LPS plus ATP induced IL-1b secretion in J774 cells.

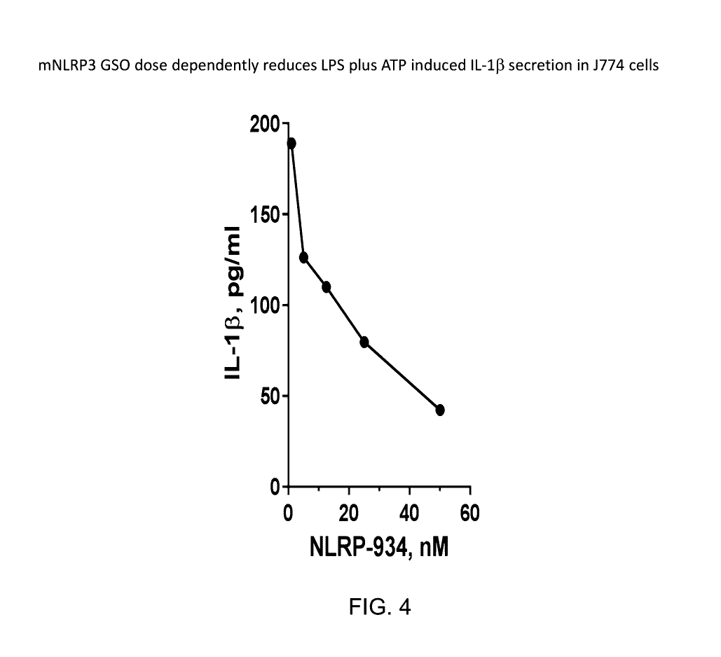

FIG. 5 demonstrates that exemplary mNLRP3 GSO specifically silences mouse NLRP3 gene expression in cell based assay.

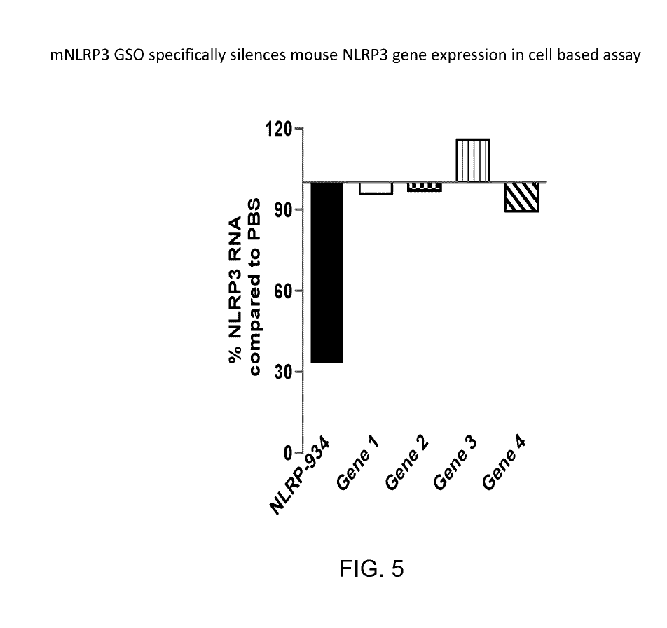

FIG. 6 depicts dose response curves of exemplary hNLRP3 GSOs in cell-based assay.

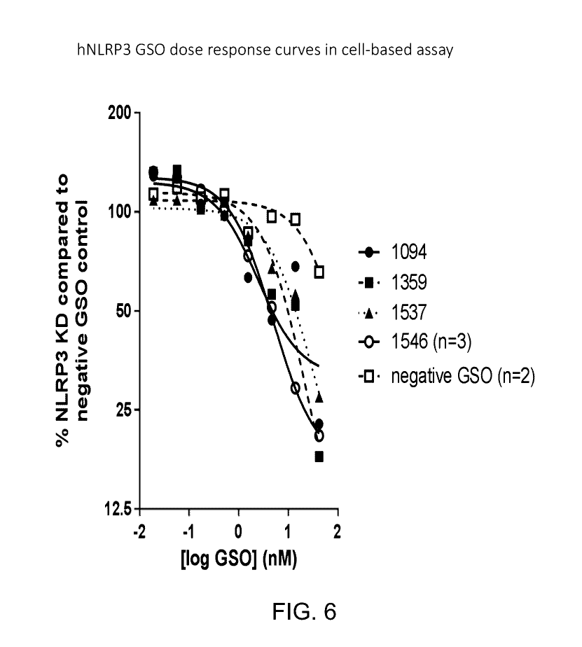

FIG. 7A and FIG. 7B depict dose response curves of exemplary hNLRP3 GSOs in THP-1 cells.

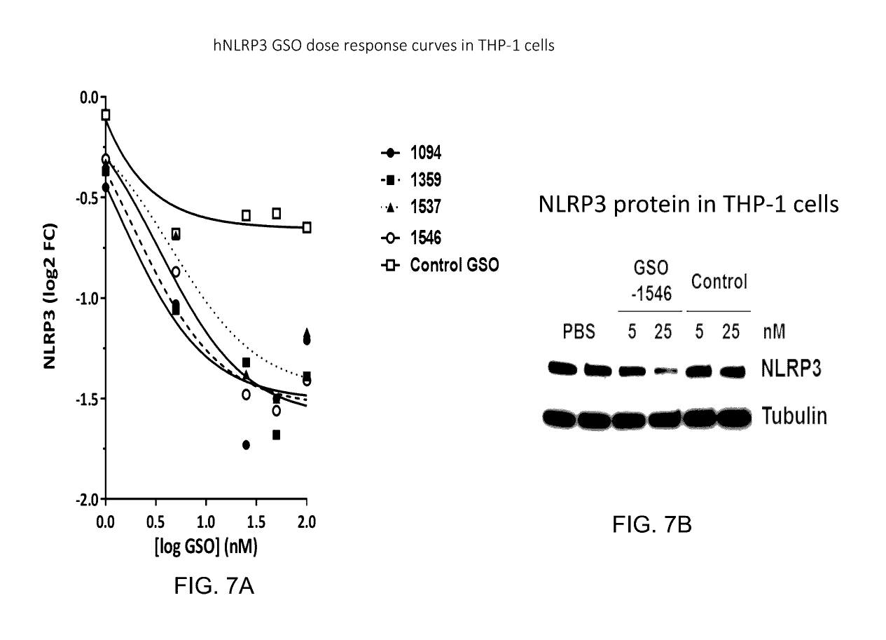

FIG. 8A and FIG. 8B demonstrate that exemplary hNLRP3 GSOs inhibit LPS/ATP-induced cytokine secretion in THP-1 cells.

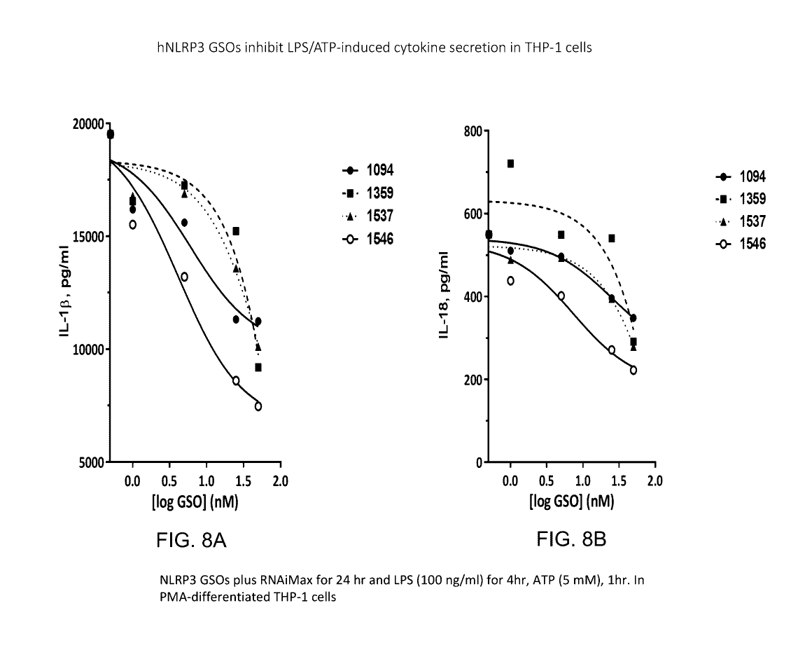

FIG. 9 depicts dose response curves of exemplary hNLRP3 GSOs in human PBMCs.

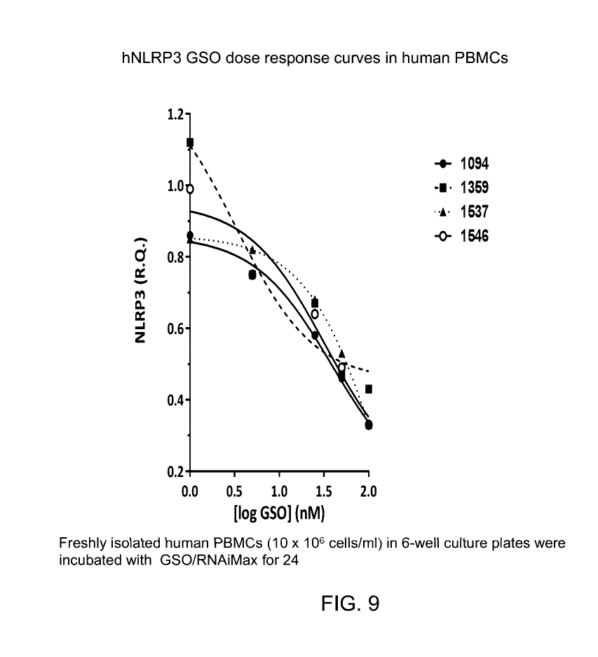

FIG. 10A and FIG. 10B demonstrate that exemplary hNLRP3 GSOs inhibit LPS/ATP-induced cytokine secretion in human PBMCs.

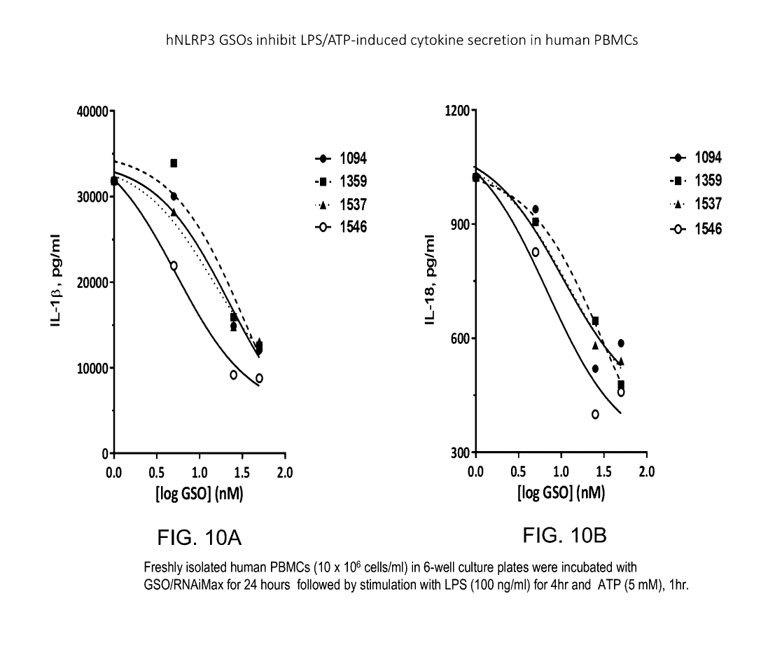

FIG. 11A through FIG. 11C demonstrate the effects of exemplary mNLRP3 GSO in an animal model of interstitial cystitis on bladder weight, urine IL-1β and urine IL-18.

FIG. 12A through FIG. 12D demonstrate the effects of exemplary mNLRP3 GSO in an animal model of interstitial cystitis on bladder inflammasome gene expression.

FIG. 13 demonstrates the effects of exemplary mNLRP3 GSO in an animal model of interstitial cystitis on bladder inflammasome gene expression.

FIG. 14 demonstrates the effects of exemplary mNLRP3 GSO in an animal model of interstitial cystitis on bladder weight.

FIG. 15A and FIG. 15B demonstrate the effects of exemplary mNLRP3 GSO in an animal model of interstitial cystitis on bladder weight, urine IL-1β.

FIG. 16A through FIG. 16D demonstrate the effects of exemplary mNLRP3 GSO in an animal model of experimental autoimmune uveitis.

### DETAILED DESCRIPTION OF THE PREFERRED EMBODIMENTS

The invention relates to the therapeutic and prophylactic use of gene silencing compounds to down-regulate NLRP3 mRNA or protein expression. Such molecules are useful, for example, in providing compositions for modulation of NLRP3 gene expression or for treating and/or preventing diseases and/or conditions that are capable of responding to modulation of NLRP3 gene expression in patients, subjects, animals or organisms.

The objects of the present invention, the various features thereof, as well as the invention itself may be more fully understood from the following description, when read together with the accompanying drawings in which the following terms have the ascribed meaning. Unless specific definitions are provided, the nomenclature utilized in connection with, and the procedures and techniques of, analytical chemistry, synthetic organic chemistry, and medicinal and pharmaceutical chemistry described herein are those well-known and commonly used in the art. Standard techniques may be used for chemical synthesis, and chemical analysis. Where permitted, all patents, applications, published applications and other publications, GENBANK Accession Numbers and associated sequence information obtainable through databases such as National Center for Biotechnology Information (NCBI) and other data referred to throughout in the disclosure herein are incorporated by reference for the portions of the document discussed herein, as well as in their entirety.

The term “2′-O-substituted” means substitution of the 2′ position of the pentose moiety with an —O— lower alkyl group containing 1-6 saturated or unsaturated carbon atoms (for example, but not limited to, 2′-O-methyl), or with an —O-aryl or allyl group having 2-6 carbon atoms, wherein such alkyl, aryl or allyl group may be unsubstituted or may be substituted, (for example, with 2′-O-methoxyethyl, ethoxy, methoxy, halo, hydroxyl, trifluoromethyl, cyano, nitro, acyl, acyloxy, alkoxy, carboxyl, carbalkoxyl, or amino groups); or with a hydroxyl, an amino or a halo group, but not with a 2′-H group. In some embodiments the oligonucleotides of the invention include four or five 2′-O-alky nucleotides at their 5′ terminus, and/or four or five 2′-O-alky nucleotides at their 3′ terminus.

The term “3′”, when used directionally, generally refers to a region or position in a polynucleotide or oligonucleotide 3′ (toward the 3′end of the nucleotide) from another region or position in the same polynucleotide or oligonucleotide.

The term “3′ end” generally refers to the 3′ terminal nucleotide of the component oligonucleotides. “Two or more oligonucleotides linked at their 3′ ends” generally refers to a linkage between the 3′ terminal nucleotides of the oligonucleotides which may be directly via 5′, 3′ or 2′ hydroxyl groups, or indirectly, via a non-nucleotide linker. Such linkages may also be via a nucleoside, utilizing both 2′ and 3′ hydroxyl positions of the nucleoside. Such linkages may also utilize a functionalized sugar or nucleobase of a 3′terminal nucleotide.

The term “5′”, when used directionally, generally refers to a region or position in a polynucleotide or oligonucleotide 5′ (toward the 5′end of the nucleotide) from another region or position in the same polynucleotide or oligonucleotide.

The term “5′ end” generally refers to the 5′ terminal nucleotide of the component oligonucleotides. “Two or more single-stranded antisense oligonucleotides linked at their 5′ ends” generally refers to a linkage between the 5′ terminal nucleotides of the oligonucleotides which may be directly via 5′, 3′ or 2′ hydroxyl groups, or indirectly, via a non-nucleotide linker. Such linkages may also be via a nucleoside, utilizing both 2′ and 3′ hydroxyl positions of the nucleoside. Such linkages may also utilize a functionalized sugar or nucleobase of a 5′terminal nucleotide.

The term “about” generally means that the exact number is not critical. Thus, oligonucleotides having one or two fewer nucleoside residues, or from one to several additional nucleoside residues are contemplated as equivalents of each of the embodiments described above.

The term “accessible” generally means when related to a compound according to the invention, that the relevant portion of the molecule is able to be recognized by the cellular components necessary to elicit an intended response to the compound.

The term “agonist” generally refers to a substance that binds to a receptor of a cell and induces a response. An agonist often mimics the action of a naturally occurring substance such as a ligand.

The term “antigen” generally refers to a substance that is recognized and selectively bound by an antibody or by a T cell antigen receptor. Antigens may include but are not limited to peptides, proteins, lipids, carbohydrates, nucleosides, nucleotides, nucleic acids, and combinations thereof. Antigens may be natural or synthetic and generally induce an immune response that is specific for that antigen.

“Antisense activity” means any detectable or measurable activity attributable to the hybridization of a gene silencing compound to its target nucleic acid. In certain embodiments, antisense activity is a decrease in the amount or expression of a target nucleic acid or protein encoded by such target nucleic acid.

“Gene silencing compound” (also referred to herein as “GSO” or “GSOs”) means an oligomeric compound comprising two or more single stranded antisense oligonucleotides that are linked through their 5′-ends to allow the presence of two or more accessible 3′-ends. Gene silencing compounds are capable of undergoing hybridization to a target nucleic acid through hydrogen bonding. Gene silencing compounds according to the invention include, but are not limited to, antisense oligonucleotides comprising naturally occurring nucleotides, modified nucleotides, modified oligonucleotides and/or backbone modified oligonucleotides.

“Antisense inhibition” means reduction of target nucleic acid levels or target protein levels in the presence of a gene silencing compound complementary to a target nucleic acid as compared to target nucleic acid levels or target protein levels in the absence of the gene silencing compound.

“Antisense oligonucleotide” means a single-stranded oligonucleotide having a nucleobase sequence that permits hybridization to a corresponding region or segment of a target nucleic acid.

The term “biologic instability” generally refers to a molecule's ability to be degraded and subsequently inactivated in vivo. For oligonucleotides, such degradation results from exonuclease activity and/or endonuclease activity, wherein exonuclease activity refers to cleaving nucleotides from the 3′ or 5′ end of an oligonucleotide, and endonuclease activity refers to cleaving phosphodiester bonds at positions other than at the ends of the oligonucleotide.

The term “carrier” generally encompasses any excipient, diluent, filler, salt, buffer, stabilizer, solubilizer, oil, lipid, lipid containing vesicle, microspheres, liposomal encapsulation, or other material for use in pharmaceutical formulations. It will be understood that the characteristics of the carrier, excipient or diluent will depend on the route of administration for a particular application. The preparation of pharmaceutically acceptable formulations containing these materials is described in, for example, Remington's Pharmaceutical Sciences, 18^{th} Edition, ed. A. Gennaro, Mack Publishing Co., Easton, Pa., 1990.

The term “co-administration” or “co-administered” generally refers to the administration of at least two different substances. Co-administration refers to simultaneous administration, as well as temporally spaced order of up to several days apart, of at least two different substances in any order, either in a single dose or separate doses.

The term “in combination with” generally means administering an oligonucleotide-based compound according to the invention and another agent useful for treating the disease or condition that does not abolish the activity of the compound in the course of treating a patient. Such administration may be done in any order, including simultaneous administration, as well as temporally spaced order from a few seconds up to several days apart. Such combination treatment may also include more than a single administration of the compound according to the invention and/or independently the other agent. The administration of the compound according to the invention and the other agent may be by the same or different routes.

The term “complementary” is intended to mean the capacity for pairing between nucleobases of a first nucleic acid and a second nucleic acid.

“Contiguous nucleobases” means nucleobases immediately adjacent to each other.

The term “individual” or “subject” or “patient” generally refers to a mammal, such as a human.

“NLRP3 nucleic acid” means any nucleic acid encoding NLRP3. For example, in certain embodiments, a NLRP3 nucleic acid includes a DNA sequence encoding NLRP3, an RNA sequence transcribed from DNA encoding NLRP3 (including genomic DNA comprising introns and exons), and an mRNA sequence encoding NLRP3. “NLRP3 mRNA” means an mRNA encoding a NLRP3 protein.

“Fully complementary” or “100% complementary” means each nucleobase of a first nucleic acid has a complementary nucleobase in a second nucleic acid. In certain embodiments, a first nucleic acid is an antisense compound and a target nucleic acid is a second nucleic acid.

“Hybridization” means the annealing of complementary nucleic acid molecules. In certain embodiments, complementary nucleic acid molecules include an antisense compound and a target nucleic acid.

“Inhibiting NLRP3 mRNA or protein expression” means reducing expression of NLRP3 mRNA and/or protein levels in the presence of a gene silencing compound according to the invention as compared to expression of NLRP3 and/or protein levels in the absence of a gene silencing compound according to the invention.

The term “linear synthesis” generally refers to a synthesis that starts at one end of an oligonucleotide and progresses linearly to the other end. Linear synthesis permits incorporation of either identical or non-identical (in terms of length, base composition and/or chemical modifications incorporated) monomeric units into an oligonucleotide.

The term “mammal” is expressly intended to include warm blooded, vertebrate animals, including, without limitation, humans, non-human primates, rats, mice, cats, dogs, horses, cattle, cows, pigs, sheep and rabbits.

The term “nucleoside” generally refers to compounds consisting of a sugar, usually ribose, deoxyribose, pentose, arabinose or hexose, and a purine or pyrimidine base.

The term “nucleotide” generally refers to a nucleoside comprising a phosphorous-containing group attached to the sugar.

The term “modified nucleoside” or “nucleotide derivative” generally is a nucleoside that includes a modified heterocyclic base, a modified sugar moiety, or any combination thereof. In some embodiments, the modified nucleoside or nucleotide derivative is a non-natural pyrimidine or purine nucleoside, as herein described. For purposes of the invention, a modified nucleoside or nucleotide derivative, a pyrimidine or purine analog or non-naturally occurring pyrimidine or purine can be used interchangeably and refers to a nucleoside that includes a non-naturally occurring base and/or non-naturally occurring sugar moiety. For purposes of the invention, a base is considered to be non-natural if it is not guanine, cytosine, adenine, thymine or uracil and a sugar is considered to be non-natural if it is not β-ribo-furanoside or 2′-deoxyribo-furanoside.

The term “modified oligonucleotide” as used herein describes an oligonucleotide in which at least two of its nucleotides are covalently linked via a synthetic linkage, i.e., a linkage other than a phosphodiester linkage between the 5′ end of one nucleotide and the 3′ end of another nucleotide in which the 5′ nucleotide phosphate has been replaced with any number of chemical groups. The term “modified oligonucleotide” also encompasses 2′-O,4′-C-methylene-b-D-ribofuranosyl nucleic acids, arabinose nucleic acids, substituted arabinose nucleic acids, hexose nucleic acids, peptide nucleic acids, morpholino, and oligonucleotides having at least one nucleotide with a modified base and/or sugar, such as a 2′-O-substituted, a 5-methylcytosine and/or a 3′-O-substituted ribonucleotide.

The term “nucleic acid” encompasses a genomic region or an RNA molecule transcribed therefrom. In some embodiments, the nucleic acid is mRNA.

The term “linker” generally refers to any moiety that can be attached to an oligonucleotide by way of covalent or non-covalent bonding through a sugar, a base, or the backbone. The non-covalent linkage may be, without limitation, electrostatic interactions, hydrophobic interactions, π-stacking interactions, hydrogen bonding and combinations thereof. Non-limiting examples of such non-covalent linkage includes Watson-Crick base pairing, Hoogsteen base pairing, and base stacking. The linker can be used to attach two or more nucleosides or can be attached to the 5′ and/or 3′ terminal nucleotide in the oligonucleotide. Such linker can be either a non-nucleotide linker or a nucleoside linker.

The term “non-nucleotide linker” generally refers to a chemical moiety, other than a linkage directly between two nucleotides that can be attached to an oligonucleotide by way of covalent or non-covalent bonding. Preferably such non-nucleotide linker is from about 2 angstroms to about 200 angstroms in length, and may be either in a cis or trans orientation.

The term “internucleotide linkage” generally refer to a chemical linkage to join two nucleosides through their sugars (e.g. 3′-3′, 2′-3′, 2′-5′, 3′-5′, 5′-5′) consisting of a phosphorous atom and a charged, or neutral group (e.g., phosphodiester, phosphorothioate, phosphorodithioate or methylphosphonate) between adjacent nucleosides.

The term “oligonucleotide” refers to a polynucleoside formed from a plurality of linked nucleoside units, which may include, for example, deoxyribonucleotides or ribonucleotides, synthetic or natural nucleotides, phosphodiester or modified linkages, natural bases or modified bases natural sugars or modified sugars, or combinations of these components. The nucleoside units may be part of viruses, bacteria, cell debris or oligonucleotide-based compositions (for example, siRNA and microRNA). Such oligonucleotides can also be obtained from existing nucleic acid sources, including genomic or cDNA, but are preferably produced by synthetic methods. In certain embodiments each nucleoside unit includes a heterocyclic base and a pentofuranosyl, trehalose, arabinose, 2′-deoxy-2′-substituted nucleoside, 2′-deoxy-2′-substituted arabinose, 2′-O-substitutedarabinose or hexose sugar group. The nucleoside residues can be coupled to each other by any of the numerous known internucleoside linkages. Such internucleoside linkages include, without limitation, phosphodiester, phosphorothioate, phosphorodithioate, methylphosphonate, alkylphosphonate, alkylphosphonothioate, phosphotriester, phosphoramidate, siloxane, carbonate, carboalkoxy, acetamidate, carbamate, morpholino, borano, thioether, bridged phosphoramidate, bridged methylene phosphonate, bridged phosphorothioate, and sulfone internucleoside linkages. The term “oligonucleotide” also encompasses polynucleosides having one or more stereospecific internucleoside linkage (e.g., (R_{P})- or (S_{P})-phosphorothioate, alkylphosphonate, or phosphotriester linkages). As used herein, the terms “oligonucleotide” and “dinucleotide” are expressly intended to include polynucleosides and dinucleosides having any such internucleoside linkage, whether or not the linkage comprises a phosphate group. In certain exemplary embodiments, these internucleoside linkages may be phosphodiester, phosphorothioate or phosphorodithioate linkages, or combinations thereof. In exemplary embodiments, the nucleotides of the synthetic oligonucleotides are linked by at least one phosphorothioate internucleotide linkage. The phosphorothioate linkages may be mixed Rp and Sp enantiomers, or they may be stereoregular or substantially stereoregular in either Rp or Sp form (see Iyer et al. (1995) Tetrahedron Asymmetry 6:1051-1054). In certain embodiments, one or more of the oligonucleotides within the antisense compositions of the invention contain one or more 2′-O,4′-C-methylene-b-D-ribofuranosyl nucleic acids, wherein the ribose is modified with a bond between the 2′ and 4′ carbons, which fixes the ribose in the 3′-endo structural conformation.

The term “peptide” generally refers to oligomers or polymers of amino acids that are of sufficient length and composition to affect a biological response, for example, antibody production or cytokine activity whether or not the peptide is a hapten. The term “peptide” may include modified amino acids (whether or not naturally or non-naturally occurring), where such modifications include, but are not limited to, phosphorylation, glycosylation, pegylation, lipidization, and methylation.

The term “pharmaceutically acceptable” means a non-toxic material that does not interfere with the effectiveness of a compound according to the invention or the biological activity of a compound according to the invention.

The term “physiologically acceptable” refers to a non-toxic material that is compatible with a biological system such as a cell, cell culture, tissue, or organism. Preferably, the biological system is a living organism, such as a mammal, particularly a human.

The term “prophylactically effective amount” generally refers to an amount sufficient to prevent or reduce the development of an undesired biological effect.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an antisense compound.

“Single-stranded oligonucleotide” means an oligonucleotide which is not hybridized to a complementary strand.

“Specifically hybridizable” refers to a gene silencing compound having a sufficient degree of complementarity between an antisense oligonucleotide and a target nucleic acid to induce a desired effect, while exhibiting minimal or no effects on non-target nucleic acids under conditions in which specific binding is desired, i.e., under physiological conditions in the case of in vivo assays and therapeutic treatments.

“Targeting” or “targeted” means the process of design and selection of a gene silencing compound that will specifically hybridize to a target nucleic acid and induce a desired effect.

“Target nucleic acid,” “target RNA,” “target mRNA,” and “target RNA transcript” all refer to a nucleic acid capable of being targeted by gene silencing compounds.

“Target segment” means the sequence of nucleotides of a target nucleic acid to which a gene silencing compound is targeted. “5′ target site” refers to the 5′-most nucleotide of a target segment. “3′ target site” refers to the 3′-most nucleotide of a target segment.

The term “therapeutically effective amount” or “pharmaceutically effective amount” generally refers to an amount sufficient to affect a desired biological effect, such as a beneficial result, including, without limitation, prevention, diminution, amelioration or elimination of signs or symptoms of a disease or disorder. Thus, the total amount of each active component of the pharmaceutical composition or method is sufficient to show a meaningful patient benefit, for example, but not limited to, healing of chronic conditions characterized by immune stimulation. Thus, a “pharmaceutically effective amount” will depend upon the context in which it is being administered. A pharmaceutically effective amount may be administered in one or more prophylactic or therapeutic administrations. When applied to an individual active ingredient, administered alone, the term refers to that ingredient alone. When applied to a combination, the term refers to combined amounts of the active ingredients that result in the therapeutic effect, whether administered in combination, serially or simultaneously.

The term “treatment” generally refers to an approach intended to obtain a beneficial or desired result, which may include alleviation of symptoms, or delaying or ameliorating a disease progression.

The term “gene expression” generally refers to process by which information from a gene is used in the synthesis of a functional gene product, which may be a protein. The process may involve transcription, RNA splicing, translation, and post-translational modification of a protein, and may include mRNA, preRNA, ribosomal RNA, and other templates for protein synthesis.

In certain embodiments provided are methods, compounds, and compositions for inhibiting NLRP3 mRNA or protein expression. In certain embodiments the compounds are antisense oligonucleotides, double stranded or single-stranded siRNA compounds, or gene silencing compounds.

As used herein, gene silencing compounds according to the invention comprise two or more single-stranded antisense oligonucleotides linked at their 5′ ends, wherein the compounds have two or more accessible 3′ ends. The general structure of the oligonucleotide-based compounds of the invention may be described by the following formula I:

3′-Nn . . . N1N2N3N4-5′-X-5′-N8N7N6N5 . . . Nm-3′ (Formula I),

wherein X is a nucleotide linker or non-nucleotide linker; N1-N8, at each occurrence, is independently a nucleotide or nucleotide derivative; Nm and Nn, at each occurrence, are independently a nucleotide or nucleotide derivative; and wherein m and n are independently numbers from 0 to about 40.

The linkage at the 5′ ends of the component oligonucleotides is independent of the other oligonucleotide linkages and may be directly via 5′, 3′ or 2′ hydroxyl groups, or indirectly, via a non-nucleotide linker or a nucleoside, utilizing either the 2′ or 3′ hydroxyl positions of the nucleoside. Linkages may also utilize a functionalized sugar or nucleobase of a 5′ terminal nucleotide.

In certain embodiments provided are gene silencing compounds targeted to a human NLRP3 nucleic acid. In certain embodiments, the human NLRP3 nucleic acid is the sequence set forth in GENBANK Accession No. NM_004895.4 (incorporated herein as SEQ ID NO: 95).

| (SEQ ID NO: 95) | |
|---|---|
| 1 | gtagatgagg aaactgaagt tgaggaatag tgaagagttt gtccaatgtc atagccccgt |
| 61 | aatcaacggg acaaaaattt tcttgctgat gggtcaagat ggcatcgtga agtggttgtt |
| 121 | caccgtaaac tgtaatacaa tcctgtttat ggatttgttt gcatattttt ccctccatag |
| 181 | ggaaaccttt cttccatggc tcaggacaca ctcctggatc gagccaacag gagaactttc |
| 241 | tggtaagcat ttggctaact tttttttttt tgagatggag tcttgctgtg tcgcctaggc |
| 301 | tggagtgcag tggcgtgatc ttggctcact gcagcctcca cttcccgggt tcaatcaatt |
| 361 | ctcctacctc aacttcctga gtagctggga ttacaggcgc ccgccaccac acccggctca |
| 421 | tttttgtact tttagtagag acacagtttt gccatgttgg ccaggctggt cttgaattcc |
| 481 | tcagctcagg tgatctgcct gccttggcct ctcaaagtgc tgggattaca ggcgtgagcc |
| 541 | actgtgcccg gccttggcta acttttcaaa attaaagatt ttgacttgtt acagtcatgt |
| 601 | gacatttttt tctttctgtt tgctgagttt ttgataattt atatctctca aagtggagac |
| 661 | tttaaaaaag actcatccgt gtgccgtgtt cactgcctgg tatcttagtg tggaccgaag |
| 721 | cctaaggacc ctgaaaacag ctgcagatga agatggcaag cacccgctgc aagctggcca |
| 781 | ggtacctgga ggacctggag gatgtggact tgaagaaatt taagatgcac ttagaggact |
| 841 | atcctcccca gaagggctgc atccccctcc cgaggggtca gacagagaag gcagaccatg |
| 901 | tggatctagc cacgctaatg atcgacttca atggggagga gaaggcgtgg gccatggccg |
| 961 | tgtggatctt cgctgcgatc aacaggagag acctttatga gaaagcaaaa agagatgagc |
| 1021 | cgaagtgggg ttcagataat gcacgtgttt cgaatcccac tgtgatatgc caggaagaca |
| 1081 | gcattgaaga ggagtggatg ggtttactgg agtacctttc gagaatctct atttgtaaaa |
| 1141 | tgaagaaaga ttaccgtaag aagtacagaa agtacgtgag aagcagattc cagtgcattg |
| 1201 | aagacaggaa tgcccgtctg ggtgagagtg tgagcctcaa caaacgctac acacgactgc |
| 1261 | gtctcatcaa ggagcaccgg agccagcagg agagggagca ggagcttctg gccatcggca |
| 1321 | agaccaagac gtgtgagagc cccgtgagtc ccattaagat ggagttgctg tttgaccccg |
| 1381 | atgatgagca ttctgagcct gtgcacaccg tggtgttcca gggggcggca gggattggga |
| 1441 | aaacaatcct ggccaggaag atgatgttgg actgggcgtc ggggacactc taccaagaca |
| 1501 | ggtttgacta tctgttctat atccactgtc gggaggtgag ccttgtgaca cagaggagcc |
| 1561 | tgggggacct gatcatgagc tgctgccccg acccaaaccc acccatccac aagatcgtga |
| 1621 | gaaaaccctc cagaatcctc ttcctcatgg acggcttcga tgagctgcaa ggtgcctttg |
| 1681 | acgagcacat aggaccgctc tgcactgact ggcagaaggc cgagcgggga gacattctcc |
| 1741 | tgagcagcct catcagaaag aagctgcttc ccgaggcctc tctgctcatc accacgagac |
| 1801 | ctgtggccct ggagaaactg cagcacttgc tggaccatcc tcggcatgtg gagatcctgg |
| 1861 | gtttctccga ggccaaaagg aaagagtact tcttcaagta cttctctgat gaggcccaag |
| 1921 | ccagggcagc cttcagtctg attcaggaga acgaggtcct cttcaccatg tgcttcatcc |
| 1981 | ccctggtctg ctggatcgtg tgcactggac tgaaacagca gatggagagt ggcaagagcc |
| 2041 | ttgcccagac atccaagacc accaccgcgg tgtacgtctt cttcctttcc agtttgctgc |
| 2101 | agccccgggg agggagccag gagcacggcc tctgcgccca cctctggggg ctctgctctt |
| 2161 | tggctgcaga tggaatctgg aaccagaaaa tcctgtttga ggagtccgac ctcaggaatc |
| 2221 | atggactgca gaaggcggat gtgtctgctt tcctgaggat gaacctgttc caaaaggaag |
| 2281 | tggactgcga gaagttctac agcttcatcc acatgacttt ccaggagttc tttgccgcca |
| 2341 | tgtactacct gctggaagag gaaaaggaag gaaggacgaa cgttccaggg agtcgtttga |
| 2401 | agcttcccag ccgagacgtg acagtccttc tggaaaacta tggcaaattc gaaaaggggt |
| 2461 | atttgatttt tgttgtacgt ttcctctttg gcctggtaaa ccaggagagg acctcctact |
| 2521 | tggagaagaa attaagttgc aagatctctc agcaaatcag gctggagctg ctgaaatgga |
| 2581 | ttgaagtgaa agccaaagct aaaaagctgc agatccagcc cagccagctg gaattgttct |
| 2641 | actgtttgta cgagatgcag gaggaggact tcgtgcaaag ggccatggac tatttcccca |
| 2701 | agattgagat caatctctcc accagaatgg accacatggt ttcttccttt tgcattgaga |
| 2761 | actgtcatcg ggtggagtca ctgtccctgg ggtttctcca taacatgccc aaggaggaag |
| 2821 | aggaggagga aaaggaaggc cgacaccttg atatggtgca gtgtgtcctc ccaagctcct |
| 2881 | ctcatgctgc ctgttctcat ggattggtga acagccacct cacttccagt ttttgccggg |
| 2941 | gcctcttttc agttctgagc accagccaga gtctaactga attggacctc agtgacaatt |
| 3001 | ctctggggga cccagggatg agagtgttgt gtgaaacgct ccagcatcct ggctgtaaca |
| 3061 | ttcggagatt gtggttgggg cgctgtggcc tctcgcatga gtgctgcttc gacatctcct |
| 3121 | tggtcctcag cagcaaccag aagctggtgg agctggacct gagtgacaac gccctcggtg |
| 3181 | acttcggaat cagacttctg tgtgtgggac tgaagcacct gttgtgcaat ctgaagaagc |
| 3241 | tctggttggt cagctgctgc ctcacatcag catgttgtca ggatcttgca tcagtattga |
| 3301 | gcaccagcca ttccctgacc agactctatg tgggggagaa tgccttggga gactcaggag |
| 3361 | tcgcaatttt atgtgaaaaa gccaagaatc cacagtgtaa cctgcagaaa ctggggttgg |
| 3421 | tgaattctgg ccttacgtca gtctgttgtt cagctttgtc ctcggtactc agcactaatc |
| 3481 | agaatctcac gcacctttac ctgcgaggca acactctcgg agacaagggg atcaaactac |
| 3541 | tctgtgaggg actcttgcac cccgactgca agcttcaggt gttggaatta gacaactgca |
| 3601 | acctcacgtc acactgctgc tgggatcttt ccacacttct gacctccagc cagagcctgc |
| 3661 | gaaagctgag cctgggcaac aatgacctgg gcgacctggg ggtcatgatg ttctgtgaag |
| 3721 | tgctgaaaca gcagagctgc ctcctgcaga acctggggtt gtctgaaatg tatttcaatt |
| 3781 | atgagacaaa aagtgcgtta gaaacacttc aagaagaaaa gcctgagctg accgtcgtct |
| 3841 | ttgagccttc ttggtaggag tggaaacggg gctgccagac gccagtgttc tccggtccct |
| 3901 | ccagctgggg gccctcaggt ggagagagct gcgatccatc caggccaaga ccacagctct |
| 3961 | gtgatccttc cggtggagtg tcggagaaga gagcttgccg acgatgcctt cctgtgcaga |
| 4021 | gcttgggcat ctcctttacg ccagggtgag gaagacacca ggacaatgac agcatcgggt |
| 4081 | gttgttgtca tcacagcgcc tcagttagag gatgttcctc ttggtgacct catgtaatta |
| 4141 | gctcattcaa taaagcactt tctttatttt tctcttctct gtctaacttt ctttttccta |
| 4201 | tcttttttct tctttgttct gtttactttt gctcatatca tcattcccgc tatctttcta |
| 4261 | ttaactgacc ataacacaga actagttgac tatatattat gttgaaattt tatggcagct |
| 4321 | atttatttat ttaaattttt tgtaacagtt ttgttttcta ataagaaaaa tccatgcttt |
| 4381 | ttgtagctgg ttgaaaattc aggaatatgt aaaacttttt ggtatttaat taaattgatt |
| 4441 | ccttttctta attttaaaaa aaaaaaaaaa |

In certain embodiments provided are gene silencing compounds targeted to a mouse NLRP3 nucleic acid. In certain embodiments, the mouse NLRP3 nucleic acid is the sequence set forth in GENBANK Accession No. NM_145827.3 (incorporated herein as SEQ ID NO: 96).

| (SEQ ID NO: 96) | |
|---|---|
| 1 | ccaggtccta gcctcgtcac catgggttct ggtcagacac gagtcctggt gactttgtat |
| 61 | atgcgtgttc tctgtatacc acatctgatt gtgttaatgg ctttcttatt tttatctcta |
| 121 | cagaggaact tttcttccat ggctcaggac atacgtctgg atcaagctaa gagaactttc |
| 181 | tgtgtggacc taagccccga gaccctcgaa agggctgctg ctgaagatga cgagtgtccg |
| 241 | ttgcaagctg gctcagtatc tagaggacct tgaagatgtg gacctcaaga aattcaaaat |
| 301 | gcatttggaa gattacccgc ccgagaaagg ctgtatccca gtccccaggg gccagatgga |
| 361 | gaaggcagat cacttggatc tagccacact catgattgac ttcaatggcg aggagaaggc |
| 421 | ctgggccatg gctgtgtgga tctttgctgc gatcaacagg cgagacctct gggaaaaagc |
| 481 | taagaaggac cagccagagt ggaatgacac gtgtacatca cattcctcta tggtatgcca |
| 541 | ggaggacagc cttgaagaag agtggatggg tttgctggga tatctctccc gcatctccat |
| 601 | ttgtaaaaag aagaaagatt actgtaagat gtacagacga catgtgagaa gcaggttcta |
| 661 | ctctatcaag gacaggaacg cgcgtctagg tgagagtgtg gacctcaaca gtcgctacac |
| 721 | gcagctccaa ctggtcaagg agcatccaag caagcaggag cgggagcatg aactcctgac |
| 781 | catcggccgg actaaaatgc gggacagccc catgagttcc cttaagctgg agctgctgtt |
| 841 | tgagcccgag gacgggcact cggagcctgt gcacacagtg gtgttccagg gagcagcagg |
| 901 | catcgggaaa accatcctag ccaggaagat tatgttggac tgggcactgg gaaagctctt |
| 961 | caaagacaaa tttgactatt tgttctttat ccactgccga gaggtgagcc tcaggacgcc |
| 1021 | aaggagtcta gcagacctga ttgtcagctg ctggcctgac ccaaacccac cagtgtgcaa |
| 1081 | gatcctgcgc aagccttcca ggatcctctt cctcatggat ggctttgatg agctacaagg |
| 1141 | ggcctttgac gagcacattg gggaggtctg cacagactgg caaaaggctg tgcggggaga |
| 1201 | cattctgcta agcagcctca tccgaaagaa actgctgccc aaggcctctc tgctcataac |
| 1261 | gacgaggccg gtagccttgg agaaactgca gcatctcctg gaccaccccc gccatgtgga |
| 1321 | gatcctaggt ttctctgagg ccaaaaggaa ggagtatttc tttaagtatt tctccaacga |
| 1381 | gctgcaggcc cgggaggcct tcaggctgat ccaagagaat gaggtcctct ttaccatgtg |
| 1441 | cttcatcccc ctggtctgct ggattgtgtg cacggggcta aagcaacaga tggagaccgg |
| 1501 | gaagagcctg gcccagacct ccaagaccac tacggccgtc tacgtcttct tcctttccag |
| 1561 | cctgctgcaa tcccgggggg gcattgagga gcatctcttc tctgactacc tacaggggct |
| 1621 | ctgttcactg gctgcggatg gaatttggaa ccagaaaatc ctatttgagg agtgtgatct |
| 1681 | gcggaagcac ggcctgcaga agactgacgt ctccgctttc ctgaggatga acgtgttcca |
| 1741 | gaaggaagtg gactgcgaga gattctacag cttcagccac atgactttcc aggagttctt |
| 1801 | cgctgctatg tactatttgc tggaagagga ggcagagggg gagaccgtga ggaaaggacc |
| 1861 | aggaggttgt tcagatcttc tgaaccgaga cgtgaaggtc ctactagaaa attacggcaa |
| 1921 | gtttgaaaaa ggctatctga tttttgttgt ccgattcctc tttggccttg taaaccagga |
| 1981 | gagaacctct tatttggaga agaaactaag ttgcaagatc tctcagcaag tcagactgga |
| 2041 | actactgaag tggattgaag tgaaagccaa ggccaagaag ctgcagtggc agcccagcca |
| 2101 | actggaactg ttctactgcc tgtacgagat gcaggaggaa gactttgtgc agagtgccat |
| 2161 | ggaccacttt cccaaaattg agatcaacct ctctaccaga atggaccacg tggtttcctc |
| 2221 | cttttgtatt aagaactgtc atagggtcaa aacgctttcc ctgggttttt ttcacaactc |
| 2281 | gcccaaggag gaagaagaag agaggagagg aggtcgaccc ttggaccagg ttcagtgtgt |
| 2341 | tttcccagac actcatgttg cctgttcttc cagactggtg aactgctgcc tcacttctag |
| 2401 | cttctgccgt ggtctcttct caagtctaag caccaaccgg agcctcactg aactggacct |
| 2461 | cagtgacaat actctgggag acccgggcat gagggtgctg tgtgaggcac tccagcaccc |
| 2521 | aggctgtaac attcagagac tgtggttggg gcgctgcgga ctgtcccatc aatgctgctt |
| 2581 | cgacatctcc tctgtcctga gcagcagcca gaagctggtg gagctggacc tcagtgacaa |
| 2641 | tgccctgggg gactttggaa tcagattgct gtgtgtggga ctgaagcacc tgctctgcaa |
| 2701 | cctccagaaa ctgtggttgg tgagctgctg tctcacatcc gcgtgttgtc aggatctcgc |
| 2761 | attggttctg agctccaacc attctctgac cagactgtac attggagaaa atgccttggg |
| 2821 | agactcagga gtccaagttt tgtgtgaaaa gatgaaggac ccacagtgta acttgcagaa |
| 2881 | gctggggttg gtgaattccg gccttacttc aatctgttgt tcagctctga cctctgtgct |
| 2941 | caaaaccaac cagaacttca cacacctcta tctacgaagc aatgcccttg gagacacagg |
| 3001 | actcaggctc ctctgtgagg ggcttctgca cccggactgt aaactacaga tgctggaatt |
| 3061 | agacaactgc agcctcacct cacacagctg ctggaatctc tccacaattc tgacccacaa |
| 3121 | ccacagcctt cggaagctga acctgggcaa caatgatctt ggcgatctgt gcgtggtgac |
| 3181 | cctctgtgag gtgctgaaac agcagggctg cctcctgcag agcctacagt tgggtgaaat |
| 3241 | gtacttaaat cgtgaaacaa aacgtgcctt agaagcgctc caggaagaaa agcctgagct |
| 3301 | gactatagtc ttcgagattt cctggtaggc gtggaagcag gaccaccagg tgcctcggtc |
| 3361 | ctgccccaag tcctgcccca agccccagtg cgcactgctc ttcactgcta tcaagccctc |
| 3421 | cttcaccatc aggatcacag ccgaggctct tctggtatag ggtctggagc aaaggcttgt |
| 3481 | gtgggaccaa atatttttcc tcacatcgat aacgtgaaac tgccagaggc tgcccttccc |
| 3541 | atcatatcct cagtgggcaa ggtgttccct cttggtgact tcatggaaac agcttcaaga |
| 3601 | aaacgccttt tctgtcctcc cccgccctcc tcttactcct gcccctcctc ctcctcctcc |
| 3661 | tcccctcccc ccccctcctc ctccgcttct ccccccacct gtctttctct ctctgggcct |
| 3721 | ctggtttttt gacctttgcc cataccttca gtcttgtctt cctgttaact gaccatcccg |
| 3781 | cataaggagc tgcccgtggg ctagatggaa ggtttgtggc agcctctcag ctacattgtt |
| 3841 | tgtttttatt ttttaatagt tatgatttct ctttagctac ctgaaaactc agagatttat |
| 3901 | aaaacccatt tttgtattta ttgtatgttt gtactgcttt cttaatttaa aaatgtatct |
| 3961 | agaattcttt taagttattt atccaaacta ctaaaaataa atcagtttac acatttaaaa |
| 4021 | t |

Certain embodiments provide gene silencing compounds comprising two oligonucleotides each, independently, consisting of 12 to 30 nucleotides having a nucleobase sequence comprising a portion of at least 12 contiguous nucleobases complementary to an equal length portion of SEQ ID NO: 95. Certain embodiments provide compounds comprising two oligonucleotides each, independently, consisting of 15 to 25 nucleotides having a nucleobase sequence comprising a portion of at least 12 contiguous nucleobases complementary to an equal length portion of SEQ ID NO: 95. Certain embodiments provide compounds comprising a modified oligonucleotide consisting of 18 to 21 nucleotides having a nucleobase sequence comprising a portion of at least 12 contiguous nucleobases complementary to an equal length portion of SEQ ID NO: 95. In certain embodiments, the two oligonucleotide of the gene silencing compound each, independently, comprise at least 9, at least 10, at least 11, at least 12, at least 13, at least 14, at least 15, at least 16, at least 17, at least 18, or at least 19 contiguous nucleobases complementary to an equal length portion of SEQ ID NO: 95.

In certain embodiments, the two oligonucleotide of the gene silencing compound each, independently, comprise at least 9, at least 10, at least 11, at least 12, at least 13, at least 14, at least 15, at least 16, at least 17, at least 18, at least 19, at least 20, at least 21, at least 22, or at least 23, contiguous nucleobases complementary to an equal length portion of SEQ ID NO: 95.

Certain embodiments provide gene silencing compounds comprising two oligonucleotides each, independently, comprising a portion which consists of least 12 contiguous nucleobases of SEQ ID NOs: 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, or 94. In certain embodiments, the gene silencing compounds comprise two oligonucleotides each, independently, comprising a portion which consists of least 12 contiguous nucleobases of SEQ ID NOs: 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, or 94, and is at least 80% complimentary to SEQ ID NO: 95. In certain embodiments, the gene silencing compounds comprise two oligonucleotides each, independently, comprising a portion which consists of least 12 contiguous nucleobases of SEQ ID NOs: 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, or 94, and is at least 85% complimentary to SEQ ID NO: 95. In certain embodiments, the gene silencing compounds comprise two oligonucleotides each, independently, comprising a portion which consists of least 12 contiguous nucleobases of SEQ ID NOs: 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, or 94, and is at least 90% complimentary to SEQ ID NO: 95. In certain embodiments, the gene silencing compounds comprise two oligonucleotides each, independently, comprising a portion which consists of least 12 contiguous nucleobases of SEQ ID NOs: 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, or 94, and is at least 95% complimentary to SEQ ID NO: 95.

In certain embodiments, the nucleobase sequence of the oligonucleotides of the gene silencing compound are, independently, at least 90% complementary over its entire length to a nucleobase sequence of SEQ ID NO: 95. In certain embodiments, the nucleobase sequence of the oligonucleotides of the gene silencing compound are, independently, at least 95% complementary over its entire length to a nucleobase sequence of SEQ ID NO: 95. In certain embodiments, the oligonucleotides of the gene silencing compound are at least 99% complementary over its entire length to SEQ ID NO: 95. In certain embodiments, the nucleobase sequence of the oligonucleotides of the gene silencing compound are 100% complementary over its entire length to a nucleobase sequence of SEQ ID NO: 95.

Certain embodiments provide gene silencing compounds comprising two oligonucleotides each, independently, consisting of 12 to 30 nucleotides having a nucleobase sequence comprising a portion of at least 12 contiguous nucleobases complementary to an equal length portion of SEQ ID NO: 96. Certain embodiments provide compounds comprising two oligonucleotides each, independently, consisting of 15 to 25 nucleotides having a nucleobase sequence comprising a portion of at least 12 contiguous nucleobases complementary to an equal length portion of SEQ ID NO: 96. Certain embodiments provide compounds comprising a modified oligonucleotide consisting of 18 to 21 nucleotides having a nucleobase sequence comprising a portion of at least 12 contiguous nucleobases complementary to an equal length portion of SEQ ID NO: 96. In certain embodiments, the two oligonucleotide of the gene silencing compound each, independently, comprise at least 9, at least 10, at least 11, at least 12, at least 13, at least 14, at least 15, at least 16, at least 17, at least 18, at least 19, at least 20, or at least 21 contiguous nucleobases complementary to an equal length portion of SEQ ID NO: 96.

In certain embodiments, the two oligonucleotide of the gene silencing compound each, independently, comprise at least 9, at least 10, at least 11, at least 12, at least 13, at least 14, at least 15, at least 16, at least 17, at least 18, at least 19, at least 20, at least 21, at least 22, or at least 23, contiguous nucleobases complementary to an equal length portion of SEQ ID NO: 96.

Certain embodiments provide gene silencing compounds comprising two oligonucleotides each, independently, comprising a portion which consists of least 12 contiguous nucleobases of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41. In certain embodiments, the gene silencing compounds comprise two oligonucleotides each, independently, comprising a portion which consists of least 12 contiguous nucleobases of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41, and is at least 80% complimentary to SEQ ID NO: 96. In certain embodiments, the gene silencing compounds comprise two oligonucleotides each, independently, comprising a portion which consists of least 12 contiguous nucleobases of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41, and is at least 85% complimentary to SEQ ID NO: 96. In certain embodiments, the gene silencing compounds comprise two oligonucleotides each, independently, comprising a portion which consists of least 12 contiguous nucleobases of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41, and is at least 90% complimentary to SEQ ID NO: 96. In certain embodiments, the gene silencing compounds comprise two oligonucleotides each, independently, comprising a portion which consists of least 12 contiguous nucleobases of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41, and is at least 95% complimentary to SEQ ID NO: 96.

In certain embodiments, the nucleobase sequence of the oligonucleotides of the gene silencing compound are, independently, at least 90% complementary over its entire length to a nucleobase sequence of SEQ ID NO: 96. In certain embodiments, the nucleobase sequence of the oligonucleotides of the gene silencing compound are, independently, at least 95% complementary over its entire length to a nucleobase sequence of SEQ ID NO: 96. In certain embodiments, the oligonucleotides of the gene silencing compound are at least 99% complementary over its entire length to SEQ ID NO: 96. In certain embodiments, the nucleobase sequence of the oligonucleotides of the gene silencing compound are 100% complementary over its entire length to a nucleobase sequence of SEQ ID NO: 96.

In certain embodiments, the oligonucleotides of the gene silencing compound are, independently, 4 to 44 nucleotides in length. In certain embodiments, the oligonucleotides of the gene silencing compound are, independently, 12 to 30 nucleotides in length. In other words, the oligonucleotides are from 12 to 30 linked nucleobases. In other embodiments, the oligonucleotides, independently, consist of 15 to 28, 18 to 24, 19 to 22, or 20 linked nucleobases. In certain such embodiments, the oligonucleotides, independently, consist of 8, 9, 10, 11, 12, 13, 14, 15, 16, 17, 18, 19, 20, 21, 22, 23, 24, 25, 26, 27, 28, 29, 30 linked nucleobases in length, or a range defined by any two of the above values.

In certain such embodiments, the oligonucleotides are 19 linked nucleobases in length.

In certain embodiments, a target region is a structurally defined region of the target nucleic acid. For example, a target region may encompass a 3′ UTR, a 5′ UTR, an exon, an intron, an exon/intron junction, a coding region, a translation initiation region, translation termination region, or other defined nucleic acid region. The structurally defined regions for NLRP3 can be obtained by accession number from sequence databases such as NCBI and such information is incorporated herein by reference. In certain embodiments, a target region may encompass the sequence from a 5′ target site of one target segment within the target region to a 3′ target site of another target segment within the same target region.

Certain embodiments provide a composition comprising a gene silencing compound as described herein, or a salt thereof, and a pharmaceutically acceptable carrier or diluent. Certain embodiments provide a composition comprising two or more gene silencing compounds as described herein, or a salt thereof, and a pharmaceutically acceptable carrier or diluent. The two or more gene silencing compounds can inhibit the mRNA or protein expression of the same target or can inhibit the mRNA or protein expression of different targets.

In certain embodiments, gene silencing compounds according to the invention comprise two identical or different sequences linked at their 5′-5′ ends via a phosphodiester, phosphorothioate or non-nucleoside linker. Gene silencing compounds according to the invention that comprise identical sequences are able to bind to a specific mRNA via Watson-Crick hydrogen bonding interactions and inhibit mRNA and protein expression. Gene silencing compounds according to the invention that comprise different sequences are able to bind to two or more different regions of one or more mRNA targets and inhibit mRNA and protein expression. Such compounds are comprised of heteronucleotide sequences complementary to target mRNA and form stable duplex structures through Watson-Crick hydrogen bonding.

The oligonucleotides of the gene silencing compounds are linked through their 5′-ends to allow the presence of two or more accessible 3′-ends. In certain embodiments, the oligonucleotides are linked through one or more of the non-nucleotide linkers listed in Table 1. In certain embodiments, a single linker listed in Table 1 is used to link the oligonucleotides of the gene silencing compounds. In certain embodiments, the linker is small molecule linker such as glycerol or a glycerol homolog of the formula HO—(CH₂)ₒ—CH(OH)—(CH₂)ₚ—OH, wherein o and p independently are integers from 1 to about 6, from 1 to about 4 or from 1 to about 3. In some other embodiments, the small molecule linker is a derivative of 1,3-diamino-2-hydroxypropane. Some such derivatives have the formula HO—(CH₂)ₘ—C(O)NH—CH₂—CH(OH)—CH₂—NHC(O)—(CH₂)ₘ—OH, wherein m is an integer from 0 to about 10, from 0 to about 6, from 2 to about 6 or from 2 to about 4. Representative non-nucleotide linkers are set forth in Table 1.

TABLE 1
Representative Non-Nucleotide Linkers

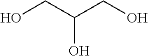

Glycerol (1,2,3-Propanetriol)

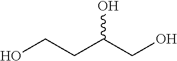

1,2,4-Butanetriol

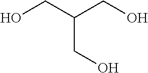

2-(hydroxymethyl)-1,3-propanediol

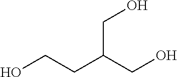

2-(hydroxymethyl)1,4-butanediol

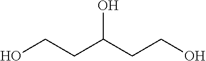

1,3,5-Pentanetriol

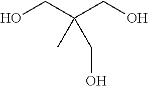

1,1,1-Tris(hydroxymethyl)ethane

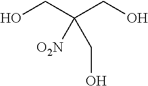

1,1,1-Tris(hydroxymethyl)nitromethane

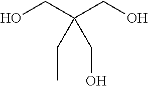

1,1,1-Tris(hydroxymethyl)propane

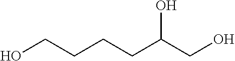

1,2,6-Hexanetriol

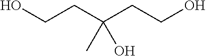

3-Methyl-1,3,5-pentanetriol

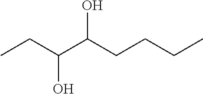

1,2,3-Heptanetriol

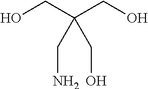

2-Amino-2-(hydroxymethyl)-1,3-propanediol

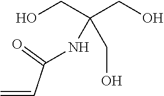

N-[Tris(hydroxymethyl)methyl]acrylamide

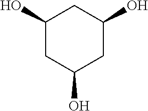

cis-1,3,5-Cyclohexanetriol

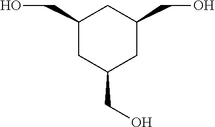 [SMILES: OC[C@H]1C[C@@H](CO)C[C@@H](CO)C1]

cis-1,3,5-Tri(hydroxymethyl)cyclohexane

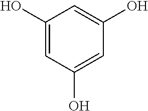

1,3,5,-Trihydroxyl-benzene

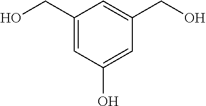 [SMILES: OCc1cc(O)cc(CO)c1]

3,5,-Di(hydroxymethyl)phenol

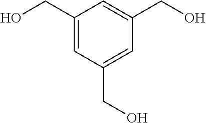 [SMILES: OCc1cc(CO)cc(CO)c1]

1,3,5,-Tri(hydroxymethyl)benzene

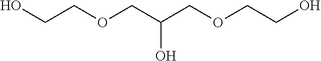 [SMILES: OCCOCC(O)COCCO]

1,3-Di(hydroxyethoxy)-2-hydroxyl-propane

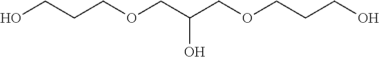 [SMILES: OCCCOCC(O)COCCCO]

1,3-Di(hydroxypropoxy)-2-hydroxyl-propane

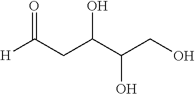

2-Deoxy-D-ribose

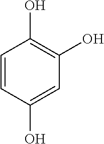

1,2,4,-Trihydroxyl-benzene

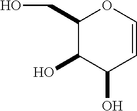

D-Galactoal

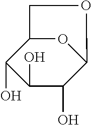 [SMILES: OC1C2COC(O2)C(O)C1O]

1,6-anhydro-β-D-Glucose

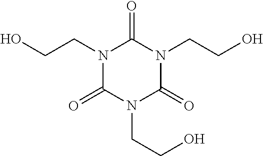 [SMILES: O=c1n(CCO)c(=O)n(CCO)c(=O)n1CCO]

1,3,5-Tris(2-hydroxyethyl)-Cyanuric acid

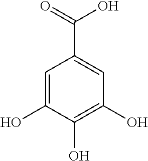 [SMILES: O=C(O)c1cc(O)c(O)c(O)c1]

Gallic acid

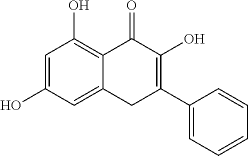 [SMILES: O=C1C(O)=C(c2ccccc2)Cc2cc(O)cc(O)c21]

3,5,7-Trihydroxyflavone

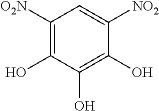 [SMILES: O=[N+]([O-])c1cc([N+](=O)[O-])c(O)c(O)c1O]

4,6-Nitropyrogallol

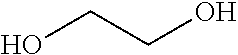

Ethylene glycol

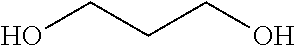

1,3-Propanediol

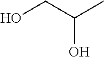

1,2-Propanediol

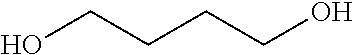

1,4-Butanediol

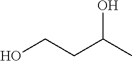

1,3-Butanediol

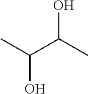

2,3-Butanediol

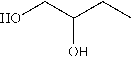

1,4-Butanediol

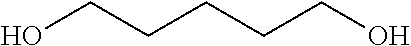

1,5-Pentanediol

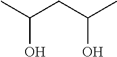

2,4-Pentanediol

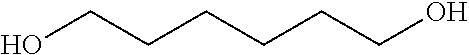

1,6-Hexanediol

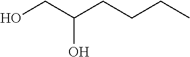

1,2-Hexanediol

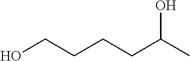

1,5-Hexanediol

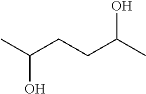

2,5-Hexanediol

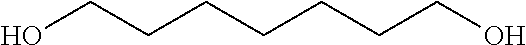

1,7-Heptanediol

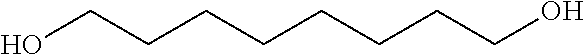

1,8-Octanediol

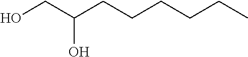

1,2-Octanediol

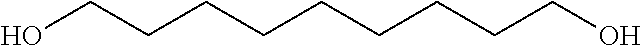 [SMILES: OCCCCCCCCCO]

1,9-Nonanediol

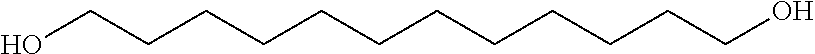 [SMILES: OCCCCCCCCCCCCO]

1,12-Dodecanediol

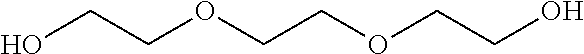

Triethylene glycol

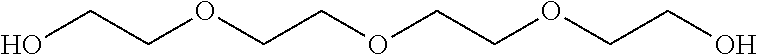 [SMILES: OCCOCCOCCOCCO]

Tetraethylene glycol

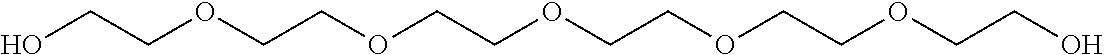 [SMILES: OCCOCCOCCOCCOCCOCCO]

Hexaethylene glycol

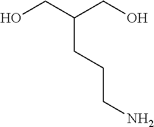

2-(1-Aminopropyl)-1,3-propanediol

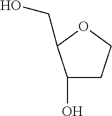

1,2-Dideoxyribose

In some embodiments, the small molecule linker is glycerol or a glycerol homolog of the formula HO—(CH₂)ₒ—CH(OH)—(CH₂)ₚ—OH, wherein o and p independently are integers from 1 to about 6, from 1 to about 4 or from 1 to about 3. In some other embodiments, the small molecule linker is a derivative of 1,3-diamino-2-hydroxypropane. Some such derivatives have the formula HO—(CH₂)ₘ—C(O)NH—CH₂—CH(OH)—CH₂—NHC(O)—(CH₂)ₘ—OH, wherein m is an integer from 0 to about 10, from 0 to about 6, from 2 to about 6 or from 2 to about 4.

In certain embodiments, the two or more oligonucleotides of the gene silencing compounds of the invention can be linked as shown in Table 2.

TABLE 2
Oligoribonucleotide Formulas II-V

| | |
|---|---|
| Formula II | 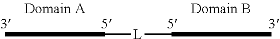 |
| Formula III | 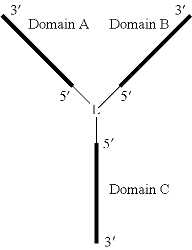 |
| Formula IV | 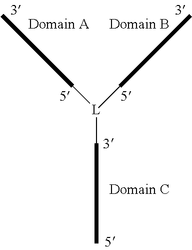 |
| Formula V | 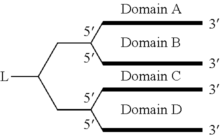 |

In certain embodiments of Formulas II and/or V, L is a linker or a nucleotide linkage and Domain A and/or Domain B are antisense oligonucleotides that are designed to selectively hybridize to the same target RNA sequence or different target RNA sequences.

In certain embodiments of Formulas II, III, IV or V, L is a linker and Domain A and/or Domain B and/or Domain C and/or Domain D are antisense oligonucleotides that are designed to selectively hybridize to the same target RNA sequence or different target RNA sequences. For example, in one embodiment, Domain A and/or Domain B and/or Domain C of Formulas II and/or III are antisense oligonucleotides that are designed to selectively hybridize to the same target RNA sequence. In this embodiment, Domain A and/or Domain B and/or Domain C can be designed to hybridize to the same region on the target RNA sequence or to different regions of the same target RNA sequence.

In a further embodiment of this aspect of the invention, Domain A, Domain B, Domain C, and Domain D are independently RNA or DNA-based oligonucleotides. In certain aspects of this embodiment, the oligonucleotides comprise mixed backbone oligonucleotides.

In another embodiment, one or more of Domain A and/or Domain B and/or Domain C and/or Domain D is an antisense oligonucleotide that is designed to selectively hybridize to one target RNA sequence and one or more of the remaining Domain A and/or Domain B and/or Domain C and/or Domain D is an antisense oligonucleotide that is designed to selectively hybridized to a different target RNA sequence.

In another embodiment, one or more of Domain A and/or Domain B and/or Domain C and/or Domain D is an RNA-based oligonucleotide hybridized to a complimentary RNA-based oligonucleotide such that the domain comprises an siRNA molecule.

These gene silencing compounds of the invention can be prepared by the art recognized methods such as phosphoramidate or H-phosphonate chemistry which can be carried out manually or by an automated synthesizer. The synthetic antisense oligonucleotides of the invention may also be modified in a number of ways without compromising their ability to hybridize to mRNA. Such modifications may include at least one internucleotide linkage of the oligonucleotide being an alkylphosphonate, phosphorothioate, phosphorodithioate, methylphosphonate, phosphate ester, alkylphosphonothioate, phosphoramidate, carbamate, carbonate, phosphate hydroxyl, acetamidate or carboxymethyl ester or a combination of these and other internucleotide linkages between the 5′ end of one nucleotide and the 3′ end of another nucleotide in which the 5′ nucleotide phosphodiester linkage has been replaced with any number of chemical groups.

The synthetic antisense oligonucleotides of the invention may comprise combinations of internucleotide linkages. For example, U.S. Pat. No. 5,149,797 describes traditional chimeric oligonucleotides having a phosphorothioate core region interposed between methylphosphonate or phosphoramidate flanking regions. Additionally, U.S. Pat. No. 5,652,356 discloses “inverted” chimeric oligonucleotides comprising one or more nonionic oligonucleotide region (e.g. alkylphosphonate and/or phosphoramidate and/or phosphotriester internucleoside linkage) flanked by one or more region of oligonucleotide phosphorothioate. Various synthetic antisense oligonucleotides with modified internucleotide linkages can be prepared according to standard methods. In certain embodiments, the phosphorothioate linkages may be mixed Rp and Sp enantiomers, or they may be made stereoregular or substantially stereoregular in either Rp or Sp form.

Other modifications of gene silencing compounds of the invention include those that are internal or at the end(s) of the oligonucleotide molecule and include additions to the molecule of the internucleoside phosphate linkages, such as cholesterol, cholesteryl, or diamine compounds with varying numbers of carbon residues between the amino groups and terminal ribose, deoxyribose and phosphate modifications which cleave, or crosslink to the opposite chains or to associated enzymes or other proteins which bind to the genome. Examples of such modified oligonucleotides include oligonucleotides with a modified base and/or sugar such as 2′-O,4′-C-methylene-b-D-ribofuranosyl, or arabinose instead of ribose, or a 3′, 5′-substituted oligonucleotide having a sugar which, at both its 3′ and 5′ positions, is attached to a chemical group other than a hydroxyl group (at its 3′ position) and other than a phosphate group (at its 5′ position).

Other examples of modifications to sugars of the oligonucleotide-based compounds of the invention include modifications to the 2′ position of the ribose moiety which include but are not limited to 2′-O-substituted with an —O-alkyl group containing 1-6 saturated or unsaturated carbon atoms, or with an —O-aryl, or —O-allyl group having 2-6 carbon atoms wherein such —O-alkyl, —O-aryl or —O-allyl group may be unsubstituted or may be substituted, for example with halo, hydroxyl, trifluoromethyl, cyano, nitro, acyl, acyloxy, alkoxy, carboxy, carbalkoxyl or amino groups. None of these substitutions are intended to exclude the presence of other residues having native 2′-hydroxyl group in the case of ribose or 2′ H— in the case of deoxyribose.

The gene silencing compounds according to the invention can comprise one or more ribonucleotides. For example, U.S. Pat. No. 5,652,355 discloses traditional hybrid oligonucleotides having regions of 2′-O-substituted ribonucleotides flanking a DNA core region. U.S. Pat. No. 5,652,356 discloses an “inverted” hybrid oligonucleotide that includes an oligonucleotide comprising a 2′-O-substituted (or 2′ OH, unsubstituted) RNA region which is in between two oligodeoxyribonucleotide regions, a structure that “inverted relative to the “traditional” hybrid oligonucleotides. Non-limiting examples of particularly useful oligonucleotides of the invention have 2′-O-alkylated ribonucleotides at their 3′, 5′, or 3′ and 5′ termini, with at least four, and in some exemplary embodiments five, contiguous nucleotides being so modified. Non-limiting examples of 2′-O-alkylated groups include 2′-O-methyl, 2′-O-ethyl, 2′-O-propyl, 2′-O-butyls and 2′-O-methoxy-ethyl.

The oligonucleotide-based compounds of the invention may conveniently be synthesized using an automated synthesizer and phosphoramidite approach further described in Example 1. In some embodiments, the oligonucleotide-based compounds of the invention are synthesized by a linear synthesis approach.

An alternative mode of synthesis is “parallel synthesis”, in which synthesis proceeds outward from a central linker moiety. A solid support attached linker can be used for parallel synthesis, as is described in U.S. Pat. No. 5,912,332. Alternatively, a universal solid support (such as phosphate attached controlled pore glass) support can be used.

Parallel synthesis of the oligonucleotide-based compounds of the invention has several advantages over linear synthesis: (1) parallel synthesis permits the incorporation of identical monomeric units; (2) unlike in linear synthesis, both (or all) the monomeric units are synthesized at the same time, thereby the number of synthetic steps and the time required for the synthesis is the same as that of a monomeric unit; and (3) the reduction in synthetic steps improves purity and yield of the final immune modulatory oligoribonucleotide product.

At the end of the synthesis by either linear synthesis or parallel synthesis protocols, the oligonucleotide-based compounds of the invention may conveniently be deprotected with concentrated ammonia solution or as recommended by the phosphoramidite supplier, if a modified nucleoside is incorporated. The product oligonucleotide-based compounds is preferably purified by reversed phase HPLC, detritylated, desalted and dialyzed.

In certain embodiments, the oligonucleotides of the gene silencing compound according to the invention are selected from the non-limiting list of the oligonucleotides shown in Table 3 below. The oligonucleotides shown in Table 3 have phosphorothioate (PS) linkages, but may also include phosphodiester linkages. Those skilled in the art will recognize, however, that other linkages, based on phosphodiester or non-phosphodiester moieties may be included.

TABLE 3

| Oligo #/SEQ | | Target | |
|---|---|---|---|
| ID NO: | Target | Site | Sequence 5′ → 3′ |
| 1 | mNLRP3 | 21 | TGTCTGACCAGAACCCATG |
| 2 | mNLRP3 | 155 | CTCTTAGCTTGATCCAGAC |
| 3 | mNLRP3 | 211 | CATCTTCAGCAGCAGCCCT |
| 4 | mNLRP3 | 242 | GATACTGAGCCAGCTTGCA |
| 5 | mNLRP3 | 349 | TGCCTTCTCCATCTGGCCC |
| 6 | mNLRP3 | 359 | CCAAGTGATCTGCCTTCTC |
| 7 | mNLRP3 | 456 | TCCCAGAGGTCTCGCCTGT |
| 8 | mNLRP3 | 485 | TCCACTCTGGCTGGTCCTT |
| 9 | mNLRP3 | 531 | CTGTCCTCCTGGCATACCA |
| 10 | mNLRP3 | 532 | GCTGTCCTCCTGGCATACC |
| 11 | mNLRP3 | 728 | GATGCTCCTTGACCAGTTG |
| 12 | mNLRP3 | 888 | CCGATGCCTGCTGCTCCCT |
| 13 | mNLRP3 | 934 | TCCCAGTGCCCAGTCCAAC |
| 14 | mNLRP3 | 936 | TTTCCCAGTGCCCAGTCCA |
| 15 | mNLRP3 | 1007 | TCCTTGGCGTCCTGAGGCT |
| 16 | mNLRP3 | 1050 | GGGTTTGGGTCAGGCCAGC |
| 17 | mNLRP3 | 1116 | AGCTCATCAAAGCCATCCA |
| 18 | mNLRP3 | 1153 | GCAGACCTCCCCAATGTGC |
| 19 | mNLRP3 | 1178 | CCCGCACAGCCTTTTGCCA |
| 20 | mNLRP3 | 1291 | GGGGTGGTCCAGGAGATGC |
| 21 | mNLRP3 | 1399 | CTCTTGGATCAGCCTGAAG |
| 22 | mNLRP3 | 1614 | GCCAGTGAACAGAGCCCCT |
| 23 | mNLRP3 | 1664 | GCAGATCACACTCCTCAAA |
| 24 | mNLRP3 | 1738 | GCAGTCCACTTCCTTCTGG |
| 25 | mNLRP3 | 1847 | CTCCTGGTCCTTTCCTCAC |
| 26 | mNLRP3 | 1848 | CCTCCTGGTCCTTTCCTCA |
| 27 | mNLRP3 | 1965 | CTCTCCTGGTTTACAAGGC |
| 28 | mNLRP3 | 2050 | GGCTTTCACTTCAATCCAC |
| 29 | mNLRP3 | 2190 | TGGTCCATTCTGGTAGAGA |
| 30 | mNLRP3 | 2320 | ACACTGAACCTGGTCCAAG |
| 31 | mNLRP3 | 2439 | TCCAGTTCAGTGAGGCTCC |
| 32 | mNLRP3 | 2505 | CCTGGGTGCTGGAGTGCCT |
| 33 | mNLRP3 | 2632 | CCCCAGGGCATTGTCACTG |
| 34 | mNLRP3 | 2810 | CTGAGTCTCCCAAGGCATT |
| 35 | mNLRP3 | 2813 | CTCCTGAGTCTCCCAAGGC |
| 36 | mNLRP3 | 2951 | AGAGGTGTGTGAAGTTCTG |
| 37 | mNLRP3 | 3014 | GGTGCAGAAGCCCCTCACA |
| 38 | mNLRP3 | 3131 | TGCCCAGGTTCAGCTTCCG |
| 39 | mNLRP3 | 3218 | CACCCAACTGTAGGCTCTG |
| 40 | mNLRP3 | 3332 | CCTGGTGGTCCTGCTTCCA |
| 41 | mNLRP3 | 3459 | AGCCTTTGCTCCAGACCCT |
| 42 | hNLRP3 | 715 | CAGGGTCCTTAGGCTTCGG |
| 43 | hNLRP3 | 743 | GCTTGCCATCTTCATCTGC |
| 44 | hNLRP3 | 796 | TTCAAGTCCACATCCTCCA |
| 45 | hNLRP3 | 827 | AGGATAGTCCTCTAAGTGC |
| 46 | hNLRP3 | 892 | GCTAGATCCACATGGTCTG |
| 47 | hNLRP3 | 985 | TTCTCATAAAGGTCTCTCC |
| 48 | hNLRP3 | 1057 | TCCTGGCATATCACAGTGG |
| 49 | hNLRP3 | 1079 | CCACTCCTCTTCAATGCTG |
| 50 | hNLRP3 | 1094 | CTCCAGTAAACCCATCCAC |
| 51 | hNLRP3 | 1191 | TCCTGTCTTCAATGCACTG |
| 52 | hNLRP3 | 1359 | GGTCAAACAGCAACTCCAT |
| 53 | hNLRP3 | 1427 | TGTTTTCCCAATCCCTGCC |
| 54 | hNLRP3 | 1434 | CCAGGATTGTTTTCCCAAT |
| 55 | hNLRP3 | 1435 | GCCAGGATTGTTTTCCCAA |
| 56 | hNLRP3 | 1537 | CTCTGTGTCACAAGGCTCA |
| 57 | hNLRP3 | 1546 | CCCAGGCTCCTCTGTGTCA |
| 58 | hNLRP3 | 1590 | GGATGGGTGGGTTTGGGTC |
| 59 | hNLRP3 | 1664 | GTCAAAGGCACCTTGCAGC |
| 60 | hNLRP3 | 1701 | CCTTCTGCCAGTCAGTGCA |
| 61 | hNLRP3 | 1702 | GCCTTCTGCCAGTCAGTGC |
| 62 | hNLRP3 | 2017 | TTGCCACTCTCCATCTGCT |
| 63 | hNLRP3 | 2018 | CTTGCCACTCTCCATCTGC |
| 64 | hNLRP3 | 2034 | ATGTCTGGGCAAGGCTCTT |
| 65 | hNLRP3 | 2046 | TGGTGGTCTTGGATGTCTG |
| 66 | hNLRP3 | 2150 | TGCAGCCAAAGAGCAGAGC |
| 67 | hNLRP3 | 2342 | CTCTTCCAGCAGGTAGTAC |
| 68 | hNLRP3 | 2429 | TTTGCCATAGTTTTCCAGA |
| 69 | hNLRP3 | 2552 | CAGCTCCAGCCTGATTTGC |
| 70 | hNLRP3 | 2773 | CCCAGGGACAGTGACTCCA |
| 71 | hNLRP3 | 2849 | GACACACTGCACCATATCA |
| 72 | hNLRP3 | 2959 | GTTAGACTCTGGCTGGTGC |
| 73 | hNLRP3 | 3012 | ACAACACTCTCATCCCTGG |
| 74 | hNLRP3 | 3121 | TGGTTGCTGCTGAGGACCA |
| 75 | hNLRP3 | 3192 | GTCCCACACACAGAAGTCT |
| 76 | hNLRP3 | 3296 | GGAATGGCTGGTGCTCAAT |
| 77 | hNLRP3 | 3315 | CCACATAGAGTCTGGTCAG |
| 78 | hNLRP3 | 3316 | CCCACATAGAGTCTGGTCA |
| 79 | hNLRP3 | 3464 | CTGATTAGTGCTGAGTACC |
| 80 | hNLRP3 | 3540 | GCAAGAGTCCCTCACAGAG |
| 81 | hNLRP3 | 3610 | AGATCCCAGCAGCAGTGTG |
| 82 | hNLRP3 | 3673 | CCCAGGTCATTGTTGCCCA |
| 83 | hNLRP3 | 3814 | GTCAGCTCAGGCTTTTCTT |
| 84 | hNLRP3 | 3815 | GGTCAGCTCAGGCTTTTCT |
| 85 | hNLRP3 | 3844 | CACTCCTACCAAGAAGGCT |
| 86 | hNLRP3 | 3849 | GTTTCCACTCCTACCAAGA |
| 87 | hNLRP3 | 3929 | TGGCCTGGATGGATCGCAG |
| 88 | hNLRP3 | 3981 | GGCAAGCTCTCTTCTCCGA |
| 89 | hNLRP3 | 4013 | GATGCCCAAGCTCTGCACA |
| 90 | hNLRP3 | 4014 | AGATGCCCAAGCTCTGCAC |
| 91 | hNLRP3 | 4015 | GAGATGCCCAAGCTCTGCA |
| 92 | hNLRP3 | 4018 | AAGGAGATGCCCAAGCTCT |
| 93 | hNLRP3 | 4101 | AGGAACATCCTCTAACTGA |
| 94 | hNLRP3 | 4265 | AGTTCTGTGTTATGGTCAG |
| 95 | mNLRP3 | 3285 | GTCAGCTCAGGCTTTTCTT |

Compound names for GSOs directed to human NLRP3 are based on the oligonucleotide target sites as depicted in SEQ ID NO: 95. Compound names for GSOs directed to mouse NLRP3 are based on their target sites of SEQ ID NO: 96. For example, a GSO comprising two copies of Oligo #13 (e.g., 3′-CAACCTGACCCGTGACCCT-5′-X-5′-TCCCAGTGCCCAGTCCAAC-3′, wherein X represents a non-nucleotidic linker) will be referred to herein, for example, as “943”, or “m943”, or “GSO 934”, “mGSO-934”, or “GSO-m934”, or “NLRP-934” or “GSO NLRP-934”. Additionally, a GSO comprising two different oligonucleotides such as Oligo #93 and Oligo #94 (e.g., 3′-AGTCAATCTCCTACAAGGA-5′-X-5′-AGTTCTGTGTTATGGTCAG-3′, wherein X represents a non-nucleotidic linker) will be referred to herein, for example, as “4101/4265”, “GSO 4101/4265”, or “NLRP-4101/4265” or “GSO NLRP-4101/4265”.

Certain embodiments provide gene silencing compounds comprising two oligonucleotides independently selected from the oligonucleotides listed in Table 3. In certain embodiments, the gene silencing compounds comprise two oligonucleotides each, independently, comprising the sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, or 94, or combinations thereof. In certain embodiments, the gene silencing compounds comprise two oligonucleotides each, independently, comprising the sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41, or combinations thereof. In certain embodiments, the gene silencing compounds comprise two oligonucleotides each, independently, comprising the sequence of SEQ ID NO: 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, or 94, or combinations thereof. In certain embodiments, the oligonucleotides of the gene silencing compound are the same. In certain embodiments, the oligonucleotides of the gene silencing compounds are different.

In certain embodiments, the invention provides a composition comprising a gene silencing compound according to the invention and one or more vaccines, antigens, antibodies, cytotoxic agents, chemotherapeutic agents (both traditional chemotherapy and modern targeted therapies), kinase inhibitors, allergens, antibiotics, agonist, antagonist, antisense oligonucleotides, ribozymes, RNAi molecules, siRNA molecules, miRNA molecules, aptamers, proteins, gene therapy vectors, DNA vaccines, adjuvants, co-stimulatory molecules or combinations thereof.

In certain embodiments, the invention provides a method for inhibiting NLRP3 mRNA or protein expression, the method comprising contacting a cell with a gene silencing compound according to the invention. In certain embodiments, the cell can be contacted with two or more gene silencing compounds targeting different regions of NLRP3.

In certain embodiments, gene silencing compounds according to the invention are useful in treating and/or preventing diseases wherein inhibiting NLRP3 expression would be beneficial.

Certain embodiments further provide a method to reduce NLRP3 mRNA or protein expression in an animal comprising administering to the animal a gene silencing compound or composition as described herein to reduce NLRP3 mRNA or protein expression in the animal. In certain embodiments, the animal is a human. In certain embodiments, reducing NLRP3 mRNA or protein expression prevents, treats, ameliorates, or slows progression of disease. In certain embodiments two or more gene silencing compounds targeting different regions of NLRP3 can be administered.

In certain embodiments provided are methods for treating diseases or disorders comprising administering to the animal a gene silencing compound or composition as described herein to reduce NLRP3 mRNA or protein expression in the animal. In certain embodiments, the animal is a human. In certain embodiments two or more gene silencing compounds targeting different regions of NLRP3 can be administered.

In certain embodiments provided are methods, compounds, and compositions for the treatment, prevention, or amelioration of diseases, disorders, and conditions associated with NLRP3 in an individual in need thereof. Also contemplated are methods and compounds for the preparation of a medicament for the treatment, prevention, or amelioration of a disease, disorder, or condition associated with NLRP3. In certain embodiments two or more gene silencing compounds targeting different regions of NLRP3 can be administered.

NLRP3 associated diseases, disorders, and conditions include, but are not limited to, familial cold autoinflammatory syndrome (FCAS), Muckle-Wells syndrome (MWS), chronic infantile neurological cutaneous and articular (CINCA) syndrome, neonatal-onset multisystem inflammatory disease (NOMID), interstitial cystitis/bladder pain syndrome (IC/BPS) multiple sclerosis, rheumatoid arthritis, gout, Alzheimer's disease, allergy and asthma, inflammatory bowel disease, atherosclerosis, type II diabetes, uveitis, hypertension, psoriasis, obesity, chronic obstructive pulmonary disease, nonalcoholic steatohepatitis, mucositis, Parkinson's disease, asbestosis, hepatomas, mesothelioma, chronic kidney disease, Schnitzler syndrome, cellulitis, conjunctivitis, dry eye syndrome, pyoderma gangrenosum, PAPA syndrome (pyogenic arthritis, pyoderma gangrenosum and acne) and any other disease, disorder or condition that would benefit from the modulation of NLRP3 mRNA or protein expression.

In certain embodiments provided are NLRP3 gene silencing compounds for use in treating, preventing, or ameliorating a NLRP3 associated disease. In certain embodiments, NLRP3 gene silencing compounds are capable of inhibiting the expression of NLRP3 mRNA and/or NLRP3 protein in a cell, tissue, or animal.

Certain embodiments provide methods comprising administering to an animal a gene silencing compounds as described herein. In certain embodiments two or more gene silencing compounds targeting different regions of NLRP3 can be administered.

Also provided are methods and gene silencing compounds for the preparation of a medicament for the treatment, prevention, or amelioration of disease.

Certain embodiments provide the use of gene silencing compounds as described herein in the manufacture of a medicament for treating, ameliorating, or preventing disease.

Certain embodiments provide gene silencing compounds as described herein for use in treating, preventing, or ameliorating disease as described herein by combination therapy with an additional agent or therapy as described herein. Agents or therapies can be co-administered or administered concomitantly.

Certain embodiments provide the use of a gene silencing compound as described herein in the manufacture of a medicament for treating, preventing, or ameliorating disease as described herein by combination therapy with an additional agent or therapy as described herein. Agents or therapies can be co-administered or administered concomitantly.

Certain embodiments provide the use of a gene silencing compound as described herein in the manufacture of a medicament for treating, preventing, or ameliorating disease as described herein in a patient who is subsequently administered an additional agent or therapy as described herein.

In any of the methods according to the invention, the gene silencing compound according to the invention can variously act by producing direct gene expression modulation effects alone and/or in combination with any other agent useful for treating or preventing the disease or condition that does not diminish the gene expression modulation effect of the gene silencing compound according to the invention. In any of the methods according to the invention, the agent(s) useful for treating or preventing the disease or condition includes, but is not limited to, vaccines, antigens, antibodies, preferably monoclonal antibodies, cytotoxic agents, kinase inhibitors, allergens, antibiotics, siRNA molecules, antisense oligonucleotides, TLR antagonist (e.g. antagonists of TLR3 and/or TLR7 and/or antagonists of TLR8 and/or antagonists of TLR9), chemotherapeutic agents (both traditional chemotherapy and modern targeted therapies), targeted therapeutic agents, activated cells, peptides, proteins, gene therapy vectors, peptide vaccines, protein vaccines, DNA vaccines, adjuvants, and co-stimulatory molecules (e.g. cytokines, chemokines, protein ligands, trans-activating factors, peptides or peptides comprising modified amino acids), or combinations thereof. Alternatively, the gene silencing compound according to the invention can be administered in combination with other compounds (for example lipids or liposomes) to enhance the specificity or magnitude of the gene expression modulation of the oligonucleotide-based compound according to the invention.

In any of the methods according to the invention, administration of gene silencing compounds according to the invention, alone or in combination with any other agent, can be by any suitable route, including, without limitation, intramuscular, parenteral, mucosal, oral, sublingual, intratumoral, transdermal, topical, inhalation, intrathecal, intranasal, aerosol, intraocular, intratracheal, intrarectal, vaginal, by gene gun, dermal patch or in eye drop or mouthwash form. In any of the methods according to the invention, administration of gene silencing compounds according to the invention, alone or in combination with any other agent, can be directly to a tissue or organ such as, but not limited to, the eye, bladder, liver, lung, kidney or lung. In certain embodiments, administration of gene silencing compounds according to the invention, alone or in combination with any other agent, is by intramuscular administration. In certain embodiments, administration of gene silencing compounds according to the invention, alone or in combination with any other agent, is by mucosal administration. In certain embodiments, administration of gene silencing compounds according to the invention, alone or in combination with any other agent, is by intraocular administration. In certain embodiments, administration of gene silencing compounds according to the invention, alone or in combination with any other agent, is by oral administration. In certain embodiments, administration of gene silencing compounds according to the invention, alone or in combination with any other agent, is by intrarectal administration. In certain embodiments, administration of gene silencing compounds according to the invention, alone or in combination with any other agent, is by intrathecal administration.

Administration of the therapeutic compositions of gene silencing compounds according to the invention can be carried out using known procedures using an effective amount and for periods of time effective to reduce symptoms or surrogate markers of the disease. For example, an effective amount of a gene silencing compound according to the invention for treating a disease and/or disorder could be that amount necessary to alleviate or reduce the symptoms, or delay or ameliorate the disease and/or disorder. In the context of administering a composition that modulates gene expression, an effective amount of a gene silencing compound according to the invention is an amount sufficient to achieve the desired modulation as compared to the gene expression in the absence of the gene silencing compound according to the invention. The effective amount for any particular application can vary depending on such factors as the disease or condition being treated, the particular compound being administered, the size of the subject, or the severity of the disease or condition. One of ordinary skill in the art can empirically determine the effective amount of a particular compound without necessitating undue experimentation.

When administered systemically, the therapeutic composition is preferably administered at a sufficient dosage to attain a blood level of gene silencing compound according to the invention from about 0.0001 micromolar to about 10 micromolar. For localized administration, much lower concentrations than this may be effective, and much higher concentrations may be tolerated. Preferably, a total dosage of gene silencing compound according to the invention ranges from about 0.001 mg per patient per day to about 200 mg per kg body weight per day. In certain embodiments, the total dosage may be 0.08, 0.16, 0.32, 0.48, 0.32, 0.64, 1, 10 or 30 mg/kg body weight administered daily, twice weekly or weekly. It may be desirable to administer simultaneously, or sequentially a therapeutically effective amount of one or more of the therapeutic compositions of the invention to an individual as a single treatment episode.

The methods according to this aspect of the invention are useful for model studies of gene expression. The methods are also useful for the prophylactic or therapeutic treatment of human or animal disease. For example, the methods are useful for pediatric and veterinary inhibition of gene expression applications.

The examples below are intended to further illustrate certain preferred embodiments of the invention, and are not intended to limit the scope of the invention.

### Examples

### Cell Culture Conditions and Reagents:

Cell lysis buffer, NLRP3 and α/β-tubulin antibodies were from Cell Signaling Technology (Danvers, Mass.). Anti-rabbit IgG-horse radish peroxidase (HRP) conjugate was from Santa Cruz Biotechnology, Inc. (Santa Cruz, Calif.). Bio-Rad protein reagent, Ready Gels, Laemmli sample buffer and PVDF membranes were from BioRad Laboratories (Hercules, Calif.), whereas Western Lightning Plus Chemiluminescence kit was from Perkin Elmer Life Sciences (Waltham, Mass.). RNeasy kits and Taqman gene expression assays and PCR reagents were purchased from Qiagen and ThermoFisher Scientific, respectively. HyBlot CL autoradiography film was purchased from Denville Scientific (Metuchen, N.J.). Human and mouse IL-18 and IL-1β ELISA kits were purchased from R&D Systems (Minneapolis, Minn.). ATP was purchased from Invivogen (San Diego, Calif.). All other chemicals and reagents were purchased either from Sigma (St. Louis, Mo.).

Murine macrophage-like cells, J774A.1 (American Type Culture Collection, Rockville, Md.) were cultured in Dulbecco's modified Eagle's medium supplemented with 10% heat-inactivated defined FBS (Hyclone) and antibiotics (100 IU/mL of penicillin G/streptomycin). All other culture reagents were purchased from Mediatech (Gaithersburg, Md.).

### Preparation of GSO/Lipid Complexes

For all gene silencing experiments, culture medium without antibiotics was used. For primary screens, exemplary GSOs shown in Table 4 were transfected at 5 and 25 nM final concentration while for dose-response curve experiments, the GSOs were serially diluted starting at 50 or 100 nM. For transfection, appropriate GSO concentrations were prepared either in culture medium (no serum) or Opti-MEM just before use, mixed with lipofectamine RNAiMax (final concentration, 3 μl/ml) and incubated at room temperature for 20 minutes.

TABLE 4

| GSO | GSO sequence |
|---|---|
| 242 | 3′-ACGTTCGACCGAGTCATAG-5′-X- |
| | 5′-GATACTGAGCCAGCTTGCA-3′ |
| 934 | 3′-CAACCTGACCCGTGACCCT-5′-X- |
| | 5′-TCCCAGTGCCCAGTCCAAC-3′ |
| 1664 | 3′-AAACTCCTCACACTAGACG-5′-X- |
| | 5′-GCAGATCACACTCCTCAAA-3′ |
| 531 | 3′-ACCATACGGTCCTCCTGTC-5′-X- |
| | 5′-CTGTCCTCCTGGCATACCA-3′ |
| 155 | 3′-CAGACCTAGTTCGATTCTC-5′-X- |
| | 5′-CTCTTAGCTTGATCCAGAC-3′ |
| 21 | 3′-GTACCCAAGACCAGTCTGT-5′-X- |
| | 5′-TGTCTGACCAGAACCCATG-3′ |
| 2050 | 3′-CACCTAACTTCACTTTCGG-5′-X- |
| | 5′-GGCTTTCACTTCAATCCAC-3′ |
| 1738 | 3′-GGTCTTCCTTCACCTGACG-5′-X- |
| | 5′ GCAGTCCACTTCCTTCTGG-3′ |
| 1399 | 3′-GAAGTCCGACTAGGTTCTC-5′-X- |
| | 5′-CTCTTGGATCAGCCTGAAG-3′ |
| 1965 | 3′-CGGAACATTTGGTCCTCTC-5′-X- |
| | 5′-CTCTCCTGGTTTACAAGGC-3′ |
| 532 | 3′-CCATACGGTCCTCCTGTCG-5′-X- |
| | 5′-GCTGTCCTCCTGGCATACC-3′ |
| 936 | 3′-ACCTGACCCGTGACCCTTT-5′-X- |
| | 5′-TTTCCCAGTGCCCAGTCCA-3′ |
| 3459 | 3′TCCCAGACCTCGTTTCCGA-5′-X- |
| | 5′-AGCCTTTGCTCCAGACCCT-3′ |
| 2320 | 3′-GAACCTGGTCCAAGTCACA-5′-X- |
| | 5′-ACACTGAACCTGGTCCAAG-3′ |
| 1094 | 3′-CACCTACCCAAATGACCTC-5′-X- |
| | 5′-CTCCAGTAAACCCATCCAC-3′ |
| 1359 | 3′-TACCTCAACGACAAACTGG-5′-X- |
| | 5′-GGTCAAACAGCAACTCCAT-3′ |
| 1537 | 3′-ACTCGGAACACTGTGTCTC-5′-X- |
| | 5′-CTCTGTGTCACAAGGCTCA-3′ |
| 1546 | 3′-ACTGTGTCTCCTCGGACCC-5′-X- |
| | 5′-CCCAGGCTCCTCTGTGTCA-3′ |

where X is glycerol.
Monitoring Gene Expression in J774, THP-1 or PBMCs Treated with GSOs

For gene silencing, J774 cells were plated overnight at a concentration of 0.3×10⁶ cells/ml in 12-well culture plates. Media was changed the next morning and the GSO/lipid complexes were added and cells were incubated at 37° C. for 24 hours.

THP-1 (1 million cells/ml in 12-well plate) cells were differentiated with phorbol myristate acetate (PMA, 500 ng/ml) for 3 hr, washed and resuspended in RPMI complete medium and incubated overnight. GSO/lipid complexes were added the next day and incubation continued for 24 hours.

Freshly isolated human PBMCs (10×10⁶ cells/nil) in 6-well culture plates were incubated with the GSO/lipid for 24 hours.

At the end of the experiment, media was removed and the pelleted cells were lysed and homogenized using a QIAshredder Kit.

### RNA Analysis

RNA was isolated using the Qiagen RNeasy Mini Kit, following the manufacturer's protocol and reverse transcribed using a High-Capacity cDNA Reverse Transcription Kit. Real Time PCR was performed on the cDNAs generated, using TaqMan Fast Universal PCR Master Mix and probes (Applied Biosystems, Carlsbad, Calif.) specific for mouse (Mm00840904_m1) or human (Hs00918082_m1) NLRP3 on StepOnePlus TaqMan Real-Time PCR System. Target mRNA levels in the samples were normalized using peptidylprolyl isomerase B, PPIB (Mm00478295_m1 and Hs00168719_m1 for mouse and human, respectively) as an endogenous control. The expression data are shown either as relative quantities or log 2FC (fold control) of NLRP3 in the samples treated with GSOs compared with a PBS control.

### Western Blotting and Quantitation of Protein Expression

For monitoring protein expression, cells were harvested at the end of the experiment, washed with chilled PBS containing protease inhibitors and then suspended in cell lysis buffer with protease inhibitors. Cells were lysed on ice for 15 minutes, sonicated briefly and centrifuged at 14000 g for 20 minutes. The supernatants were transferred to fresh vials and stored at −70° C. till further use. Protein concentration in the samples was measured by the method of Bradford using the Bio-Rad protein assay. Samples (20-30 μg/lane) were subjected to gel electrophoresis using 10% or 4-15% gradient Tris-HCl Ready gels, and western blotted onto PVDF membranes. After blocking in 5% non-fat milk in PBS-Tween 20 for 1 h, the membranes were incubated overnight with the appropriate primary antibody. Labeled proteins were visualized by the enhanced chemiluminescence method using HRP-coupled secondary antibodies and quantitated using a Scion Image Analysis Program (Scion Corp., Fredrick, Md.). For re-probing of Western blots, the blots were washed in PBS, incubated for 30 min in the stripping buffer (Thermo Scientific). They were then washed thoroughly in PBST and probed with another primary antibody using the protocol described above.

### Functional Inhibition Studies

Following incubation of cells with GSOs for 24 hours, media was changed and cells were primed with lipopolysaccharide (100 ng ml) for 4 hours and stimulated with ATP (5 mM) for 1 hour. Culture supernatants were analyzed for IL-1β and IL-18 secretion by ELISA. Cell lysates were processed for RNA isolation and quantitative real-time PCR analysis for NLRP3.

mNLRP3 GSO in an Animal Model of Interstitial Cystitis

1.) Acute CYP-IC Model Treated with GSO s.c.

Interstitial cystitis was induced in four groups (n=10) of female CD1 mice, 7 to 9 weeks of age (Charles River Laboratories, Wilmington, Mass.) by intraperitoneal injection with 200 mg/kg of cyclophosphamide (Sigma, St. Louis, Mo.) diluted in PBS. The mice were treated by subcutaneous injection of different doses of NLRP GSO or PBS as vehicle 1 h post-cyclophosphamide administration. Five naïve CD1 mice of the same sex and age, without any treatment, were used as controls.

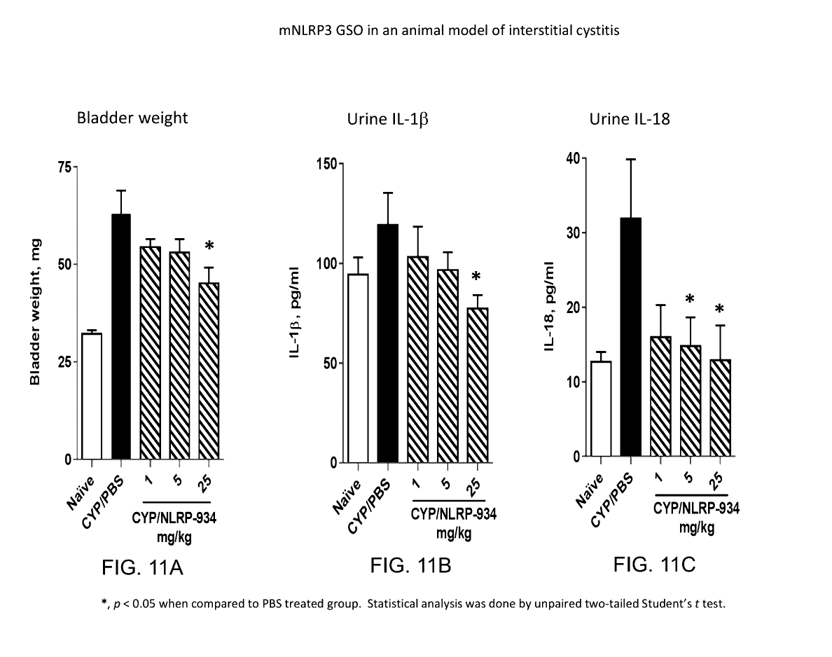

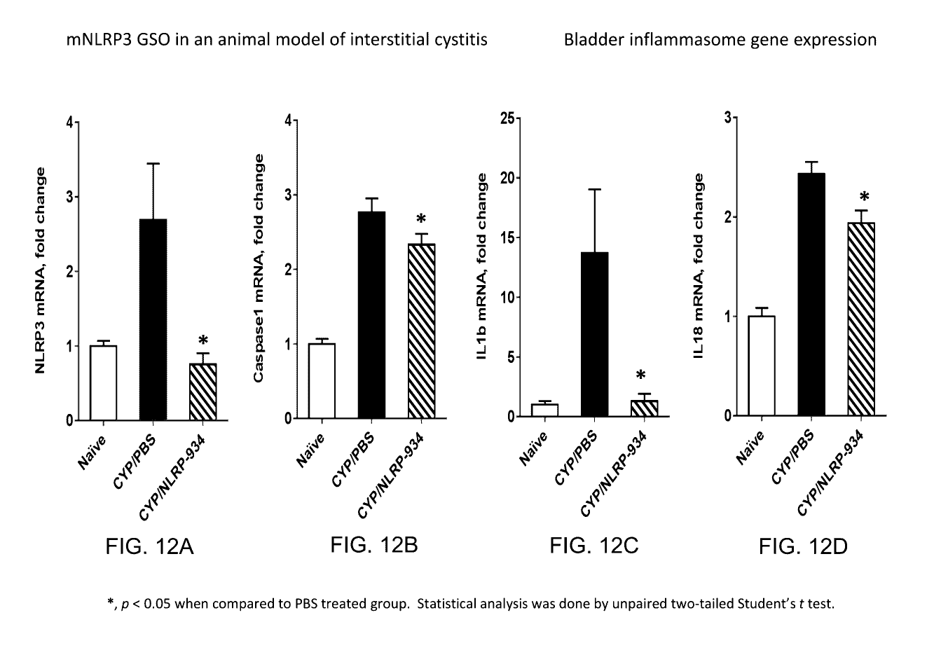

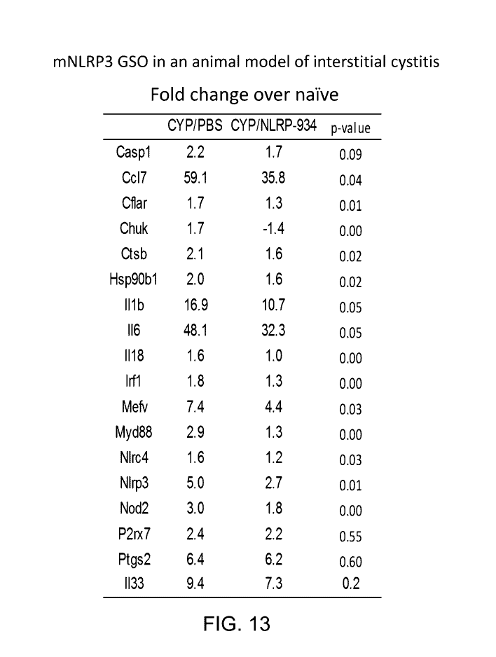

All mice were sacrificed at 24 h post disease induction. Urine samples were collected and stored at −20° C. for cytokine assay later. Bladders were collected, weighed, and stored in 10% neutral buffered formalin for histology process, or stored in RNALater for gene expression analysis. Results are shown in FIGS. 11-13.

2.) Chronic CYP-IC Model Treated with GSO s.c.

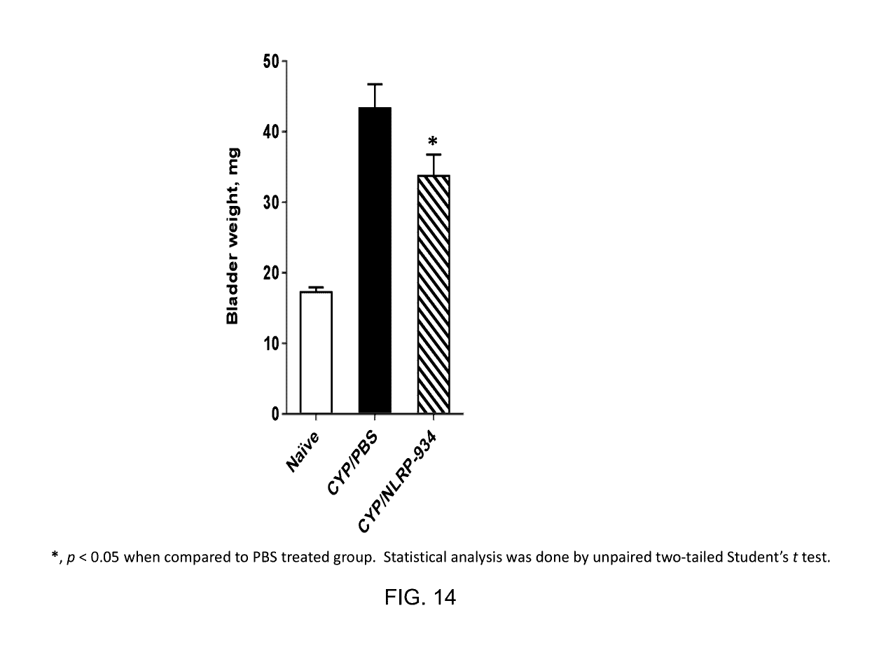

Interstitial cystitis was induced in two groups (n=10) of female CD1 mice, 7 to 9 weeks of age by intraperitoneal injection with 150 mg/kg of cyclophosphamide at day 0, 1 and 3. The mice were treated by subcutaneous injection of 25 mg/kg of NLRP GSO or PBS as vehicle 1, 2 and 3 days post-cyclophosphamide administration. Five naïve CD1 mice of the same sex and age, without treatment, were used as controls. Results are shown in FIG. 14.

3.) Acute CYP-IC Model Treated with Intrabladder Instillation of GSO

Interstitial cystitis was induced in four groups (n=5) of female CD1 mice, 7 to 9 weeks of age by intraperitoneal injection with 200 mg/kg of cyclophosphamide diluted in PBS. The mice were treated by intra-bladder (i.b.) instillation of different doses of NLRP GSO or PBS as vehicle 1 h post-cyclophosphamide administration. Five naïve CD1 mice of the same sex and age, without any treatment, were used as controls.

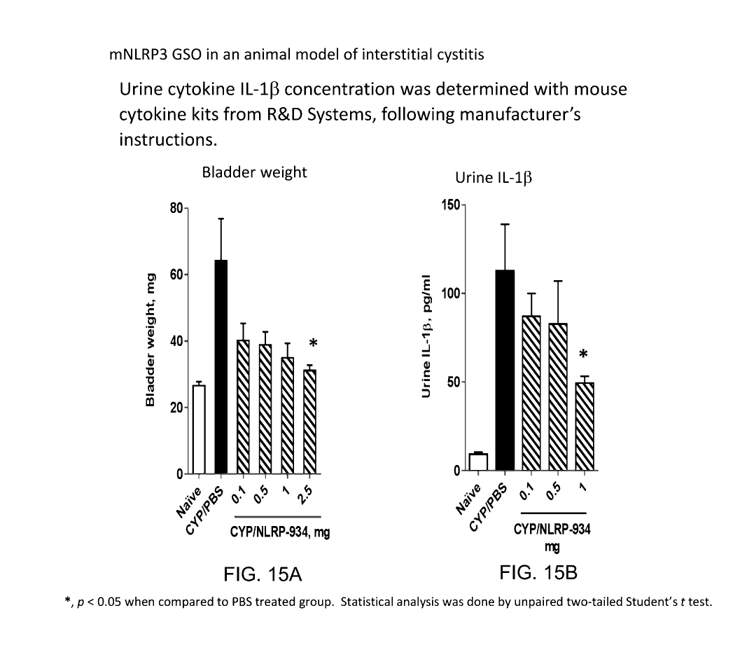

All mice were sacrificed at 24 h post disease induction. Urine samples were collected and stored at −20° C. for cytokine assay later. Bladders were collected, weighed, and stored in 10% neutral buffered formalin for histology. Results are shown in FIGS. 15A and B.

mNLRP3 GSO in an Animal Model of Experimental Autoimmune Uveitis

To induce experimental autoimmune uveitis, 6 to 7 week old male B10-RIII mice (Jackson Laboratories, Bar Harbor, Me., USA) were injected at base of the tail and two thighs with 100 μg of IRBP161-180 peptide (AnaSpec, San Jose, Calif.) and 1 mg of bovine eye homogenate (InVision BioResources, Seattle, Wash.) emulsified 1:1 vol/vol in complete Freund's adjuvant (Sigma, St Lois, Mo.) supplemented with Mycobacterium tuberculosis (Voigt Global Distribution Inc., Lawrence, Kans.) to 10 mg/ml concentration. Four hours after the first immunization, mice with injected i.p. with 0.5 μg of pertussis Toxin (List Biological Laboratories, Campbell, Calif.).

All mice received a boost immunization of 100 μg of IRBP/1 mg bovine eye homogenate emulsified 1:1 vol/vol in incomplete Freund's adjuvant (Sigma) on day 7.

Immunized mice were treated by subcutaneous injection of 15 mg/kg of NLRP GSO or PBS as vehicle at day 8, 10 and 13. Five naïve B10.RIII mice of the same sex and age, without any treatment, were used as controls.

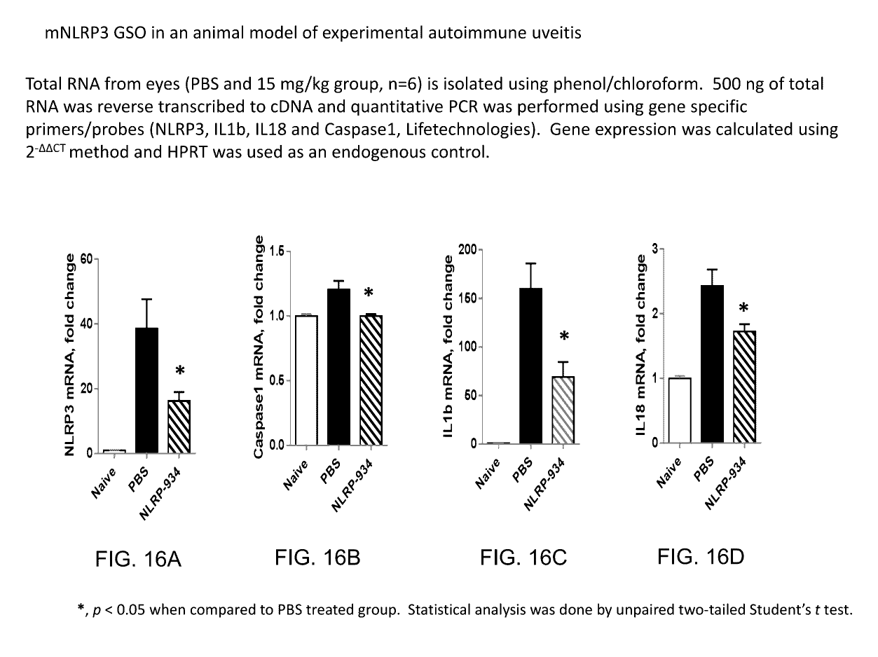

All mice were sacrificed at day 14 after blood samples were collected. The left eyes from each mouse were collected and stored in 10% neutral buffered formalin for histology process, and right eyes are stored in RNA Later for gene expression analysis. Results are shown in FIG. 16A-16D.

### EQUIVALENTS

Those skilled in the art will recognize, or be able to ascertain, using no more than routine experimentation, numerous equivalents to the specific substances and procedures described herein. For example, antisense oligonucleotides that overlap with the oligonucleotides may be used. Such equivalents are considered to be within the scope of this invention, and are covered by the following claims.

While this invention has been particularly shown and described with references to preferred embodiments thereof, it will be understood by those skilled in the art that various changes in form and details may be made therein without departing from the scope of the invention encompassed by the appended claims.

What is claimed is:

1. A synthetic oligonucleotide compound comprising two single stranded antisense oligonucleotides that are linked through their 5′-ends to allow the presence of two or more accessible 3′-ends, each oligonucleotide, independently, comprises 12 to 30 nucleotides having a nucleobase sequence comprising a portion of at least 12 contiguous nucleobases complementary to an equal length portion of SEQ ID NO: 95 or SEQ ID NO: 96.

2. The compound according to claim 1, wherein the oligonucleotides comprise the same sequence.

3. The compound according to claim 1, wherein the oligonucleotides are each, independently, between 15 to 25 nucleotides in length.

4. The compound according to claim 1, wherein the nucleobase sequence of each oligonucleotide is, independently, at least 90% complementary over its entire length to a nucleobase sequence of SEQ ID NO: 95.

5. The compound according to claim 1, wherein the nucleobase sequence of each oligonucleotide is, independently, at least 90% complementary over its entire length to a nucleobase sequence of SEQ ID NO: 96.

6. The compound according to claim 1, wherein each oligonucleotide, independently, comprises a portion of at least 12 contiguous nucleobases of SEQ ID NO: 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, or 94.

7. The compound according to claim 1, wherein each oligonucleotide, independently, comprises a portion of at least 12 contiguous nucleobases of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41.

8. The compound according to claim 1, wherein each oligonucleotide, independently, comprises a portion of at least 12 contiguous nucleobases of SEQ ID NO: 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, or 94, and is at least 80% complimentary to its target site with SEQ ID NO: 95.

9. The compound according to claim 1, wherein each oligonucleotide, independently, comprises a portion of at least 12 contiguous nucleobases of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41, and is at least 80% complimentary to its target site with SEQ ID NO: 96.

10. A composition comprising a compound according to claim 1 and a pharmaceutically acceptable carrier.

11. A synthetic oligonucleotide compound comprising two single stranded antisense oligonucleotides that are linked through their 5′-ends to allow the presence of two or more accessible 3′-ends, wherein the oligonucleotides, independently, comprise a sequence selected from SEQ ID NOs: 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, or 94.

12. The compound according to claim 11, wherein the oligonucleotides comprise the same sequence.

13. A composition comprising a compound according to claim 11 and a pharmaceutically acceptable carrier.

14. A synthetic oligonucleotide compound comprising two single stranded antisense oligonucleotides that are linked through their 5′-ends to allow the presence of two or more accessible 3′-ends, wherein the oligonucleotides, independently, comprise a sequence selected from SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41.

15. The compound according to claim 14, wherein the oligonucleotides comprise the same sequence.

16. A composition comprising a compound according to claim 14 and a pharmaceutically acceptable carrier.

17. The composition according to claim 10, further one or more vaccines, antigens, antibodies, cytotoxic agents, chemotherapeutic agents, kinase inhibitors, allergens, antibiotics, agonist, antagonist, antisense oligonucleotides, ribozymes, RNAi molecules, siRNA molecules, miRNA molecules, aptamers, proteins, gene therapy vectors, DNA vaccines, adjuvants, co-stimulatory molecules or combinations thereof.

18. The composition according to claim 13, further one or more vaccines, antigens, antibodies, cytotoxic agents, chemotherapeutic agents, kinase inhibitors, allergens, antibiotics, agonist, antagonist, antisense oligonucleotides, ribozymes, RNAi molecules, siRNA molecules, miRNA molecules, aptamers, proteins, gene therapy vectors, DNA vaccines, adjuvants, co-stimulatory molecules or combinations thereof.

19. The composition according to claim 16, further one or more vaccines, antigens, antibodies, cytotoxic agents, chemotherapeutic agents, kinase inhibitors, allergens, antibiotics, agonist, antagonist, antisense oligonucleotides, ribozymes, RNAi molecules, siRNA molecules, miRNA molecules, aptamers, proteins, gene therapy vectors, DNA vaccines, adjuvants, co-stimulatory molecules or combinations thereof.

20. A method for inhibiting NLRP3 mRNA or protein expression, the method comprising contacting a cell with at least one compound according to claim 1.

21. The method according to claim 20, wherein the cell is contacted with two or more compounds targeting different regions of NLRP3.

22. A method for inhibiting NLRP3 mRNA or protein expression, the method comprising contacting a cell with at least one compound according to claim 11.

23. The method according to claim 22, wherein the cell is contacted with two or more compounds targeting different regions of NLRP3.

24. A method for inhibiting NLRP3 mRNA or protein expression, the method comprising contacting a cell with at least one compound according to claim 14.

25. The method according to claim 24, wherein the cell is contacted with two or more compounds targeting different regions of NLRP3.

26. A method for the treatment of a disease, disorder, or condition associated with NLRP3 in an individual in need thereof, the method comprising administering a compound according to claim 1.

27. A method for the treatment of a disease, disorder, or condition associated with NLRP3 in an individual in need thereof, the method comprising administering a compound according to claim 11.

28. A method for the treatment of a disease, disorder, or condition associated with NLRP3 in an individual in need thereof, the method comprising administering a compound according to claim 14.


SEQUENCE LISTING (97 sequences)

SEQ ID NO: 1 - DNA, 19 bp, Artificial Sequence
  Synthetic oligonucleotide
tgtctgaccagaacccatg

SEQ ID NO: 2 - DNA, 19 bp, Artificial Sequence
  Synthetic oligonucleotide
ctcttagcttgatccagac

SEQ ID NO: 3 - DNA, 19 bp, Artificial Sequence
  Synthetic oligonucleotide
catcttcagcagcagccct

SEQ ID NO: 4 - DNA, 19 bp, Artificial Sequence
  Synthetic oligonucleotide
gatactgagccagcttgca

SEQ ID NO: 5 - DNA, 19 bp, Artificial Sequence
  Synthetic oligonucleotide
tgccttctccatctggccc

SEQ ID NO: 6 - DNA, 19 bp, Artificial Sequence
  Synthetic oligonucleotide
ccaagtgatctgccttctc

SEQ ID NO: 7 - DNA, 19 bp, Artificial Sequence
  Synthetic oligonucleotide
tcccagaggtctcgcctgt

SEQ ID NO: 8 - DNA, 19 bp, Artificial Sequence
  Synthetic oligonucleotide
tccactctggctggtcctt

SEQ ID NO: 9 - DNA, 19 bp, Artificial Sequence
  Synthetic oligonucleotide
ctgtcctcctggcatacca

SEQ ID NO: 10 - DNA, 19 bp, Artificial Sequence
  Synthetic oligonucleotide
gctgtcctcctggcatacc

SEQ ID NO: 11 - DNA, 19 bp, Artificial Sequence
  Synthetic oligonucleotide
gatgctccttgaccagttg

SEQ ID NO: 12 - DNA, 19 bp, Artificial Sequence
  Synthetic oligonucleotide
ccgatgcctgctgctccct

SEQ ID NO: 13 - DNA, 19 bp, Artificial Sequence
  Synthetic oligonucleotide
tcccagtgcccagtccaac

SEQ ID NO: 14 - DNA, 19 bp, Artificial Sequence
  Synthetic oligonucleotide
tttcccagtgcccagtcca

SEQ ID NO: 15 - DNA, 19 bp, Artificial Sequence
  Synthetic oligonucleotide
tccttggcgtcctgaggct

SEQ ID NO: 16 - DNA, 19 bp, Artificial Sequence
  Synthetic oligonucleotide
gggtttgggtcaggccagc

SEQ ID NO: 17 - DNA, 19 bp, Artificial Sequence
  Synthetic oligonucleotide
agctcatcaaagccatcca

SEQ ID NO: 18 - DNA, 19 bp, Artificial Sequence
  Synthetic oligonucleotide
gcagacctccccaatgtgc

SEQ ID NO: 19 - DNA, 19 bp, Artificial Sequence
  Synthetic oligonucleotide
cccgcacagccttttgcca

SEQ ID NO: 20 - DNA, 19 bp, Artificial Sequence
  Synthetic oligonucleotide
ggggtggtccaggagatgc

SEQ ID NO: 21 - DNA, 19 bp, Artificial Sequence
  Synthetic oligonucleotide
ctcttggatcagcctgaag

SEQ ID NO: 22 - DNA, 19 bp, Artificial Sequence
  Synthetic oligonucleotide
gccagtgaacagagcccct

SEQ ID NO: 23 - DNA, 19 bp, Artificial Sequence
  Synthetic oligonucleotide
gcagatcacactcctcaaa

SEQ ID NO: 24 - DNA, 19 bp, Artificial Sequence
  Synthetic oligonucleotide
gcagtccacttccttctgg

SEQ ID NO: 25 - DNA, 19 bp, Artificial Sequence
  Synthetic oligonucleotide
ctcctggtcctttcctcac

SEQ ID NO: 26 - DNA, 19 bp, Artificial Sequence
  Synthetic oligonucleotide
cctcctggtcctttcctca

SEQ ID NO: 27 - DNA, 19 bp, Artificial Sequence
  Synthetic oligonucleotide
ctctcctggtttacaaggc

SEQ ID NO: 28 - DNA, 19 bp, Artificial Sequence
  Synthetic oligonucleotide
ggctttcacttcaatccac

SEQ ID NO: 29 - DNA, 19 bp, Artificial Sequence
  Synthetic oligonucleotide
tggtccattctggtagaga

SEQ ID NO: 30 - DNA, 19 bp, Artificial Sequence
  Synthetic oligonucleotide
acactgaacctggtccaag

SEQ ID NO: 31 - DNA, 19 bp, Artificial Sequence
  Synthetic oligonucleotide
tccagttcagtgaggctcc

SEQ ID NO: 32 - DNA, 19 bp, Artificial Sequence
  Synthetic oligonucleotide
cctgggtgctggagtgcct

SEQ ID NO: 33 - DNA, 19 bp, Artificial Sequence
  Synthetic oligonucleotide
ccccagggcattgtcactg

SEQ ID NO: 34 - DNA, 19 bp, Artificial Sequence
  Synthetic oligonucleotide
ctgagtctcccaaggcatt

SEQ ID NO: 35 - DNA, 19 bp, Artificial Sequence
  Synthetic oligonucleotide
ctcctgagtctcccaaggc

SEQ ID NO: 36 - DNA, 19 bp, Artificial Sequence
  Synthetic oligonucleotide
agaggtgtgtgaagttctg

SEQ ID NO: 37 - DNA, 19 bp, Artificial Sequence
  Synthetic oligonucleotide
ggtgcagaagcccctcaca

SEQ ID NO: 38 - DNA, 19 bp, Artificial Sequence
  Synthetic oligonucleotide
tgcccaggttcagcttccg

SEQ ID NO: 39 - DNA, 19 bp, Artificial Sequence
  Synthetic oligonucleotide
cacccaactgtaggctctg

SEQ ID NO: 40 - DNA, 19 bp, Artificial Sequence
  Synthetic oligonucleotide
cctggtggtcctgcttcca

SEQ ID NO: 41 - DNA, 19 bp, Artificial Sequence
  Synthetic oligonucleotide
agcctttgctccagaccct

SEQ ID NO: 42 - DNA, 19 bp, Artificial Sequence
  Synthetic oligonucleotide
cagggtccttaggcttcgg

SEQ ID NO: 43 - DNA, 19 bp, Artificial Sequence
  Synthetic oligonucleotide
gcttgccatcttcatctgc

SEQ ID NO: 44 - DNA, 19 bp, Artificial Sequence
  Synthetic oligonucleotide
ttcaagtccacatcctcca

SEQ ID NO: 45 - DNA, 19 bp, Artificial Sequence
  Synthetic oligonucleotide
aggatagtcctctaagtgc

SEQ ID NO: 46 - DNA, 19 bp, Artificial Sequence
  Synthetic oligonucleotide
gctagatccacatggtctg

SEQ ID NO: 47 - DNA, 19 bp, Artificial Sequence
  Synthetic oligonucleotide
ttctcataaaggtctctcc

SEQ ID NO: 48 - DNA, 19 bp, Artificial Sequence
  Synthetic oligonucleotide
tcctggcatatcacagtgg

SEQ ID NO: 49 - DNA, 19 bp, Artificial Sequence
  Synthetic oligonucleotide
ccactcctcttcaatgctg

SEQ ID NO: 50 - DNA, 19 bp, Artificial Sequence
  Synthetic oligonucleotide
ctccagtaaacccatccac

SEQ ID NO: 51 - DNA, 19 bp, Artificial Sequence
  Synthetic oligonucleotide
tcctgtcttcaatgcactg

SEQ ID NO: 52 - DNA, 19 bp, Artificial Sequence
  Synthetic oligonucleotide
ggtcaaacagcaactccat

SEQ ID NO: 53 - DNA, 19 bp, Artificial Sequence
  Synthetic oligonucleotide
tgttttcccaatccctgcc

SEQ ID NO: 54 - DNA, 19 bp, Artificial Sequence
  Synthetic oligonucleotide
ccaggattgttttcccaat

SEQ ID NO: 55 - DNA, 19 bp, Artificial Sequence
  Synthetic oligonucleotide
gccaggattgttttcccaa

SEQ ID NO: 56 - DNA, 19 bp, Artificial Sequence
  Synthetic oligonucleotide
ctctgtgtcacaaggctca

SEQ ID NO: 57 - DNA, 19 bp, Artificial Sequence
  Synthetic oligonucleotide
cccaggctcctctgtgtca

SEQ ID NO: 58 - DNA, 19 bp, Artificial Sequence
  Synthetic oligonucleotide
ggatgggtgggtttgggtc

SEQ ID NO: 59 - DNA, 19 bp, Artificial Sequence
  Synthetic oligonucleotide
gtcaaaggcaccttgcagc

SEQ ID NO: 60 - DNA, 19 bp, Artificial Sequence
  Synthetic oligonucleotide
ccttctgccagtcagtgca

SEQ ID NO: 61 - DNA, 19 bp, Artificial Sequence
  Synthetic oligonucleotide
gccttctgccagtcagtgc

SEQ ID NO: 62 - DNA, 19 bp, Artificial Sequence
  Synthetic oligonucleotide
ttgccactctccatctgct

SEQ ID NO: 63 - DNA, 19 bp, Artificial Sequence
  Synthetic oligonucleotide
cttgccactctccatctgc

SEQ ID NO: 64 - DNA, 19 bp, Artificial Sequence
  Synthetic oligonucleotide
atgtctgggcaaggctctt

SEQ ID NO: 65 - DNA, 19 bp, Artificial Sequence
  Synthetic oligonucleotide
tggtggtcttggatgtctg

SEQ ID NO: 66 - DNA, 19 bp, Artificial Sequence
  Synthetic oligonucleotide
tgcagccaaagagcagagc

SEQ ID NO: 67 - DNA, 19 bp, Artificial Sequence
  Synthetic oligonucleotide
ctcttccagcaggtagtac

SEQ ID NO: 68 - DNA, 19 bp, Artificial Sequence
  Synthetic oligonucleotide
tttgccatagttttccaga

SEQ ID NO: 69 - DNA, 19 bp, Artificial Sequence
  Synthetic oligonucleotide
cagctccagcctgatttgc

SEQ ID NO: 70 - DNA, 19 bp, Artificial Sequence
  Synthetic oligonucleotide
cccagggacagtgactcca

SEQ ID NO: 71 - DNA, 19 bp, Artificial Sequence
  Synthetic oligonucleotide
gacacactgcaccatatca

SEQ ID NO: 72 - DNA, 19 bp, Artificial Sequence
  Synthetic oligonucleotide
gttagactctggctggtgc

SEQ ID NO: 73 - DNA, 19 bp, Artificial Sequence
  Synthetic oligonucleotide
acaacactctcatccctgg

SEQ ID NO: 74 - DNA, 19 bp, Artificial Sequence
  Synthetic oligonucleotide
tggttgctgctgaggacca

SEQ ID NO: 75 - DNA, 19 bp, Artificial Sequence
  Synthetic oligonucleotide
gtcccacacacagaagtct

SEQ ID NO: 76 - DNA, 19 bp, Artificial Sequence
  Synthetic oligonucleotide
ggaatggctggtgctcaat

SEQ ID NO: 77 - DNA, 19 bp, Artificial Sequence
  Synthetic oligonucleotide
ccacatagagtctggtcag

SEQ ID NO: 78 - DNA, 19 bp, Artificial Sequence
  Synthetic oligonucleotide
cccacatagagtctggtca

SEQ ID NO: 79 - DNA, 19 bp, Artificial Sequence
  Synthetic oligonucleotide
ctgattagtgctgagtacc

SEQ ID NO: 80 - DNA, 19 bp, Artificial Sequence
  Synthetic oligonucleotide
gcaagagtccctcacagag

SEQ ID NO: 81 - DNA, 19 bp, Artificial Sequence
  Synthetic oligonucleotide
agatcccagcagcagtgtg

SEQ ID NO: 82 - DNA, 19 bp, Artificial Sequence
  Synthetic oligonucleotide
cccaggtcattgttgccca

SEQ ID NO: 83 - DNA, 19 bp, Artificial Sequence
  Synthetic oligonucleotide
gtcagctcaggcttttctt

SEQ ID NO: 84 - DNA, 19 bp, Artificial Sequence
  Synthetic oligonucleotide
ggtcagctcaggcttttct

SEQ ID NO: 85 - DNA, 19 bp, Artificial Sequence
  Synthetic oligonucleotide
cactcctaccaagaaggct

SEQ ID NO: 86 - DNA, 19 bp, Artificial Sequence
  Synthetic oligonucleotide
gtttccactcctaccaaga

SEQ ID NO: 87 - DNA, 19 bp, Artificial Sequence
  Synthetic oligonucleotide
tggcctggatggatcgcag

SEQ ID NO: 88 - DNA, 19 bp, Artificial Sequence
  Synthetic oligonucleotide
ggcaagctctcttctccga

SEQ ID NO: 89 - DNA, 19 bp, Artificial Sequence
  Synthetic oligonucleotide
gatgcccaagctctgcaca

SEQ ID NO: 90 - DNA, 19 bp, Artificial Sequence
  Synthetic oligonucleotide
agatgcccaagctctgcac

SEQ ID NO: 91 - DNA, 19 bp, Artificial Sequence
  Synthetic oligonucleotide
gagatgcccaagctctgca

SEQ ID NO: 92 - DNA, 19 bp, Artificial Sequence
  Synthetic oligonucleotide
aaggagatgcccaagctct

SEQ ID NO: 93 - DNA, 19 bp, Artificial Sequence
  Synthetic oligonucleotide
aggaacatcctctaactga

SEQ ID NO: 94 - DNA, 19 bp, Artificial Sequence
  Synthetic oligonucleotide
agttctgtgttatggtcag

SEQ ID NO: 95 - DNA, 4470 bp, Artificial Sequence
  Synthetic oligonucleotide
gtagatgaggaaactgaagttgaggaatagtgaagagtttgtccaatgtcatagccccgtaatcaacgggacaaaaattttcttgctgatgggtcaagatggcatcgtgaagtggttgttcaccgtaaactgtaatacaatcctgtttatggatttgtttgcatatttttccctccatagggaaacctttcttccatggctcaggacacactcctggatcgagccaacaggagaactttctggtaagcatttggctaacttttttttttttgagatggagtcttgctgtgtcgcctaggctggagtgcagtggcgtgatcttggctcactgcagcctccacttcccgggttcaatcaattctcctacctcaacttcctgagtagctgggattacaggcgcccgccaccacacccggctcatttttgtacttttagtagagacacagttttgccatgttggccaggctggtcttgaattcctcagctcaggtgatctgcctgccttggcctctcaaagtgctgggattacaggcgtgagccactgtgcccggccttggctaacttttcaaaattaaagattttgacttgttacagtcatgtgacatttttttctttctgtttgctgagtttttgataatttatatctctcaaagtggagactttaaaaaagactcatccgtgtgccgtgttcactgcctggtatcttagtgtggaccgaagcctaaggaccctgaaaacagctgcagatgaagatggcaagcacccgctgcaagctggccaggtacctggaggacctggaggatgtggacttgaagaaatttaagatgcacttagaggactatcctccccagaagggctgcatccccctcccgaggggtcagacagagaaggcagaccatgtggatctagccacgctaatgatcgacttcaatggggaggagaaggcgtgggccatggccgtgtggatcttcgctgcgatcaacaggagagacctttatgagaaagcaaaaagagatgagccgaagtggggttcagataatgcacgtgtttcgaatcccactgtgatatgccaggaagacagcattgaagaggagtggatgggtttactggagtacctttcgagaatctctatttgtaaaatgaagaaagattaccgtaagaagtacagaaagtacgtgagaagcagattccagtgcattgaagacaggaatgcccgtctgggtgagagtgtgagcctcaacaaacgctacacacgactgcgtctcatcaaggagcaccggagccagcaggagagggagcaggagcttctggccatcggcaagaccaagacgtgtgagagccccgtgagtcccattaagatggagttgctgtttgaccccgatgatgagcattctgagcctgtgcacaccgtggtgttccagggggcggcagggattgggaaaacaatcctggccaggaagatgatgttggactgggcgtcggggacactctaccaagacaggtttgactatctgttctatatccactgtcgggaggtgagccttgtgacacagaggagcctgggggacctgatcatgagctgctgccccgacccaaacccacccatccacaagatcgtgagaaaaccctccagaatcctcttcctcatggacggcttcgatgagctgcaaggtgcctttgacgagcacataggaccgctctgcactgactggcagaaggccgagcggggagacattctcctgagcagcctcatcagaaagaagctgcttcccgaggcctctctgctcatcaccacgagacctgtggccctggagaaactgcagcacttgctggaccatcctcggcatgtggagatcctgggtttctccgaggccaaaaggaaagagtacttcttcaagtacttctctgatgaggcccaagccagggcagccttcagtctgattcaggagaacgaggtcctcttcaccatgtgcttcatccccctggtctgctggatcgtgtgcactggactgaaacagcagatggagagtggcaagagccttgcccagacatccaagaccaccaccgcggtgtacgtcttcttcctttccagtttgctgcagccccggggagggagccaggagcacggcctctgcgcccacctctgggggctctgctctttggctgcagatggaatctggaaccagaaaatcctgtttgaggagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctggaagaggaaaaggaaggaaggacgaacgttccagggagtcgtttgaagcttcccagccgagacgtgacagtccttctggaaaactatggcaaattcgaaaaggggtatttgatttttgttgtacgtttcctctttggcctggtaaaccaggagaggacctcctacttggagaagaaattaagttgcaagatctctcagcaaatcaggctggagctgctgaaatggattgaagtgaaagccaaagctaaaaagctgcagatccagcccagccagctggaattgttctactgtttgtacgagatgcaggaggaggacttcgtgcaaagggccatggactatttccccaagattgagatcaatctctccaccagaatggaccacatggtttcttccttttgcattgagaactgtcatcgggtggagtcactgtccctggggtttctccataacatgcccaaggaggaagaggaggaggaaaaggaaggccgacaccttgatatggtgcagtgtgtcctcccaagctcctctcatgctgcctgttctcatggattggtgaacagccacctcacttccagtttttgccggggcctcttttcagttctgagcaccagccagagtctaactgaattggacctcagtgacaattctctgggggacccagggatgagagtgttgtgtgaaacgctccagcatcctggctgtaacattcggagattgtggttggggcgctgtggcctctcgcatgagtgctgcttcgacatctccttggtcctcagcagcaaccagaagctggtggagctggacctgagtgacaacgccctcggtgacttcggaatcagacttctgtgtgtgggactgaagcacctgttgtgcaatctgaagaagctctggttggtcagctgctgcctcacatcagcatgttgtcaggatcttgcatcagtattgagcaccagccattccctgaccagactctatgtgggggagaatgccttgggagactcaggagtcgcaattttatgtgaaaaagccaagaatccacagtgtaacctgcagaaactggggttggtgaattctggccttacgtcagtctgttgttcagctttgtcctcggtactcagcactaatcagaatctcacgcacctttacctgcgaggcaacactctcggagacaaggggatcaaactactctgtgagggactcttgcaccccgactgcaagcttcaggtgttggaattagacaactgcaacctcacgtcacactgctgctgggatctttccacacttctgacctccagccagagcctgcgaaagctgagcctgggcaacaatgacctgggcgacctgggggtcatgatgttctgtgaagtgctgaaacagcagagctgcctcctgcagaacctggggttgtctgaaatgtatttcaattatgagacaaaaagtgcgttagaaacacttcaagaagaaaagcctgagctgaccgtcgtctttgagccttcttggtaggagtggaaacggggctgccagacgccagtgttctccggtccctccagctgggggccctcaggtggagagagctgcgatccatccaggccaagaccacagctctgtgatccttccggtggagtgtcggagaagagagcttgccgacgatgccttcctgtgcagagcttgggcatctcctttacgccagggtgaggaagacaccaggacaatgacagcatcgggtgttgttgtcatcacagcgcctcagttagaggatgttcctcttggtgacctcatgtaattagctcattcaataaagcactttctttatttttctcttctctgtctaactttctttttcctatcttttttcttctttgttctgtttacttttgctcatatcatcattcccgctatctttctattaactgaccataacacagaactagttgactatatattatgttgaaattttatggcagctatttatttatttaaattttttgtaacagttttgttttctaataagaaaaatccatgctttttgtagctggttgaaaattcaggaatatgtaaaactttttggtatttaattaaattgattccttttcttaattttaaaaaaaaaaaaaaa

SEQ ID NO: 96 - DNA, 4021 bp, Artificial Sequence
  Synthetic oligonucleotide
ccaggtcctagcctcgtcaccatgggttctggtcagacacgagtcctggtgactttgtatatgcgtgttctctgtataccacatctgattgtgttaatggctttcttatttttatctctacagaggaacttttcttccatggctcaggacatacgtctggatcaagctaagagaactttctgtgtggacctaagccccgagaccctcgaaagggctgctgctgaagatgacgagtgtccgttgcaagctggctcagtatctagaggaccttgaagatgtggacctcaagaaattcaaaatgcatttggaagattacccgcccgagaaaggctgtatcccagtccccaggggccagatggagaaggcagatcacttggatctagccacactcatgattgacttcaatggcgaggagaaggcctgggccatggctgtgtggatctttgctgcgatcaacaggcgagacctctgggaaaaagctaagaaggaccagccagagtggaatgacacgtgtacatcacattcctctatggtatgccaggaggacagccttgaagaagagtggatgggtttgctgggatatctctcccgcatctccatttgtaaaaagaagaaagattactgtaagatgtacagacgacatgtgagaagcaggttctactctatcaaggacaggaacgcgcgtctaggtgagagtgtggacctcaacagtcgctacacgcagctccaactggtcaaggagcatccaagcaagcaggagcgggagcatgaactcctgaccatcggccggactaaaatgcgggacagccccatgagttcccttaagctggagctgctgtttgagcccgaggacgggcactcggagcctgtgcacacagtggtgttccagggagcagcaggcatcgggaaaaccatcctagccaggaagattatgttggactgggcactgggaaagctcttcaaagacaaatttgactatttgttctttatccactgccgagaggtgagcctcaggacgccaaggagtctagcagacctgattgtcagctgctggcctgacccaaacccaccagtgtgcaagatcctgcgcaagccttccaggatcctcttcctcatggatggctttgatgagctacaaggggcctttgacgagcacattggggaggtctgcacagactggcaaaaggctgtgcggggagacattctgctaagcagcctcatccgaaagaaactgctgcccaaggcctctctgctcataacgacgaggccggtagccttggagaaactgcagcatctcctggaccacccccgccatgtggagatcctaggtttctctgaggccaaaaggaaggagtatttctttaagtatttctccaacgagctgcaggcccgggaggccttcaggctgatccaagagaatgaggtcctctttaccatgtgcttcatccccctggtctgctggattgtgtgcacggggctaaagcaacagatggagaccgggaagagcctggcccagacctccaagaccactacggccgtctacgtcttcttcctttccagcctgctgcaatcccgggggggcattgaggagcatctcttctctgactacctacaggggctctgttcactggctgcggatggaatttggaaccagaaaatcctatttgaggagtgtgatctgcggaagcacggcctgcagaagactgacgtctccgctttcctgaggatgaacgtgttccagaaggaagtggactgcgagagattctacagcttcagccacatgactttccaggagttcttcgctgctatgtactatttgctggaagaggaggcagagggggagaccgtgaggaaaggaccaggaggttgttcagatcttctgaaccgagacgtgaaggtcctactagaaaattacggcaagtttgaaaaaggctatctgatttttgttgtccgattcctctttggccttgtaaaccaggagagaacctcttatttggagaagaaactaagttgcaagatctctcagcaagtcagactggaactactgaagtggattgaagtgaaagccaaggccaagaagctgcagtggcagcccagccaactggaactgttctactgcctgtacgagatgcaggaggaagactttgtgcagagtgccatggaccactttcccaaaattgagatcaacctctctaccagaatggaccacgtggtttcctccttttgtattaagaactgtcatagggtcaaaacgctttccctgggtttttttcacaactcgcccaaggaggaagaagaagagaggagaggaggtcgacccttggaccaggttcagtgtgttttcccagacactcatgttgcctgttcttccagactggtgaactgctgcctcacttctagcttctgccgtggtctcttctcaagtctaagcaccaaccggagcctcactgaactggacctcagtgacaatactctgggagacccgggcatgagggtgctgtgtgaggcactccagcacccaggctgtaacattcagagactgtggttggggcgctgcggactgtcccatcaatgctgcttcgacatctcctctgtcctgagcagcagccagaagctggtggagctggacctcagtgacaatgccctgggggactttggaatcagattgctgtgtgtgggactgaagcacctgctctgcaacctccagaaactgtggttggtgagctgctgtctcacatccgcgtgttgtcaggatctcgcattggttctgagctccaaccattctctgaccagactgtacattggagaaaatgccttgggagactcaggagtccaagttttgtgtgaaaagatgaaggacccacagtgtaacttgcagaagctggggttggtgaattccggccttacttcaatctgttgttcagctctgacctctgtgctcaaaaccaaccagaacttcacacacctctatctacgaagcaatgcccttggagacacaggactcaggctcctctgtgaggggcttctgcacccggactgtaaactacagatgctggaattagacaactgcagcctcacctcacacagctgctggaatctctccacaattctgacccacaaccacagccttcggaagctgaacctgggcaacaatgatcttggcgatctgtgcgtggtgaccctctgtgaggtgctgaaacagcagggctgcctcctgcagagcctacagttgggtgaaatgtacttaaatcgtgaaacaaaacgtgccttagaagcgctccaggaagaaaagcctgagctgactatagtcttcgagatttcctggtaggcgtggaagcaggaccaccaggtgcctcggtcctgccccaagtcctgccccaagccccagtgcgcactgctcttcactgctatcaagccctccttcaccatcaggatcacagccgaggctcttctggtatagggtctggagcaaaggcttgtgtgggaccaaatatttttcctcacatcgataacgtgaaactgccagaggctgcccttcccatcatatcctcagtgggcaaggtgttccctcttggtgacttcatggaaacagcttcaagaaaacgccttttctgtcctcccccgccctcctcttactcctgcccctcctcctcctcctcctcccctccccccccctcctcctccgcttctccccccacctgtctttctctctctgggcctctggttttttgacctttgcccataccttcagtcttgtcttcctgttaactgaccatcccgcataaggagctgcccgtgggctagatggaaggtttgtggcagcctctcagctacattgtttgtttttattttttaatagttatgatttctctttagctacctgaaaactcagagatttataaaacccatttttgtatttattgtatgtttgtactgctttcttaatttaaaaatgtatctagaattcttttaagttatttatccaaactactaaaaataaatcagtttacacatttaaaat

SEQ ID NO: 97 - DNA, 19 bp, Artificial Sequence
  Synthetic oligonucleotide
gtcagctcaggcttttctt